(19) United States
(12) Patent Application Publication
(10) Pub. No.: US 2010/0077491 A1
(43) Pub. Date: Mar. 25, 2010

(54) Modulators of RNF5 and Uses Thereof

(75) Inventors: Ze'ev Ronai, La Jolla, CA (US); Agnes DeLaunay, La Jolla, CA (US); Kenneth Bromberg, New York, NY (US)

Correspondence Address: PATENT CORRESPONDENCE;ARNALL GOLDEN GREGORY LLP, 171 17TH STREET NW, SUITE 2100, ATLANTA, GA, 30363, US

(73) Assignees: Burnham Institute for Medical Research, La Jolla, CA (US); Mount Sinai School of Medicine, New York, NY (US)

(21) Appl. No.: 12/373,314

(22) PCT Filed: Jul. 12, 2007
(86) PCT No.: PCT/US07/73326

§ 371 Date: Dec. 1, 2009

Related U.S. Application Data
(60) Provisional application No. 60/807,290, filed on Jul. 13, 2006.

(51) Int. Cl. A01K 67/027; C12N 15/87; G01N 33/50; A61K 31/7088; C12Q 1/68

(52) U.S. Cl. 800/3; 800/14; 800/15; 800/18; 800/16; 800/17; 800/19; 800/21; 514/44 R; 435/6

(57) ABSTRACT

Provided herein are compositions and methods relating to the involvement of RNF5 in muscle wasting.

### CROSS-REFERENCE TO RELATED APPLICATIONS

This application claims benefit of U.S. Provisional Application No. 60/807,290, filed Jul. 13, 2006, which is hereby incorporated herein by reference in its entirety.

### STATEMENT REGARDING FEDERALLY SPONSORED RESEARCH

This invention was made with government support under Grant CA097105 awarded by the National Cancer Institute. The government has certain rights in the invention.

### BACKGROUND OF THE INVENTION

Muscular dystrophies (MD) are a group of more than 30 genetic diseases characterized by progressive weakness and degeneration of the skeletal muscles that control movement. Some forms of MD are seen in infancy or childhood, while others may not appear until middle age or later. The disorders differ in terms of the distribution and extent of muscle weakness (some forms of MD also affect cardiac muscle), age of onset, rate of progression, and pattern of inheritance.

There is no specific treatment to stop or reverse any form of MD. Treatment may include physical therapy, respiratory therapy, speech therapy, orthopedic appliances used for support, and corrective orthopedic surgery. Drug therapy includes corticosteroids to slow muscle degeneration, anticonvulsants to control seizures and some muscle activity, immunosuppressants to delay some damage to dying muscle cells, and antibiotics to fight respiratory infections. Some individuals may benefit from occupational therapy and assistive technology. Some patients may need assisted ventilation to treat respiratory muscle weakness and a pacemaker for cardiac abnormalities.

The prognosis for people with MD varies according to the type and progression of the disorder. Some cases may be mild and progress very slowly over a normal lifespan, while others produce severe muscle weakness, functional disability, and loss of the ability to walk. Some children with MD die in infancy while others live into adulthood with only moderate disability.

In muscular dystrophy (MD) the imbalance between muscle protein synthesis and degradation is an important factor leading to muscle wasting. The three major pathways of muscle proteolysis identified in skeletal muscle are: the lysosomal cathepsin pathway, the calcium-dependent calpain pathway, and the ATP-dependent ubiquitin pathway. In particular, the ubiquitin-proteasome pathway is vital for breakdown of muscle contractile proteins and can induce muscle loss if inappropriately activated. Thus, needed in the art are compositions and methods for modulating the ubiquitin-proteasome pathway for the treatment and prevention of muscular dystrophy.

### BRIEF SUMMARY OF THE INVENTION

In accordance with the purpose of this invention, as embodied and broadly described herein, this invention relates to the ubiquitin ligase referred to as RING [Really Interesting New Gene] finger 5 (RNF5). For example, provided herein are animal models wherein RNF5 expression is altered. Also provided are methods comprising modulating RNF5 levels and/or activity. Also provided are methods comprising detecting RNF5.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the RING finger domain of RNF5 exhibits high similarity to other known RING finger proteins such as c-Cb1 and BRCA1, while the C-terminal region contains a unique membrane anchoring domain. In addition, the entire protein sequence is highly conserved through evolution. Homologs to RNF5 are found in Drosophila, Arabidopsis and in mammals.

FIG. 2 shows organization of the promoter used for the inducible expression of the transgenic RNF5. Our plan is to replace the β-globin promoter, which has been used for the current observations with a muscle specific promoter including desmin.

FIG. 3 shows RNF5 transgene expression after Doxycyclin (Dox) treatment of double transgenic (RNF5+, reverse tetracycline transactivator (rtTA)) mice. Doxycyclin was added to the drinking water at a final concentration of 2 mg/ml in 5% sucrose during 4 weeks. Two sets of control were used: double transgenic animals not submitted to Doxycyclin and single transgenic animals (RNF5+) submitted to doxycyclin treatment. Western (Skeletal muscle) or IP Western blots (Kidney, Heart) were then performed on the lysates obtained from the corresponding organs using RNF5 polyclonal antibody.

FIG. 4A shows double transgenic animal (left) compared with control mice. Both were provided with doxycyclin in the drinking water for period of 2 weeks before picture was taken. FIG. 4B demonstrates the hunch back (Kyphosis) phenotype seen in the RNF5 transgenic (tg) within 3-5 weeks following administration of Dox.

FIG. 5 shows histological examination of the muscles from RNF5 Tg (right panels) compared with control Tg animals, following 6 weeks of treatment with Dox. Upper figures depicts higher magnification. The histological analysis was performed in collaboration with Diane Shelton at UCSD.

FIG. 6 shows the pTRE2hyg2-HA construct used to make RNF-5 transgenic mice. A nucleic acid encoding RNF5 was cloned into the construct at the multiple cloning site.

FIG. 7 shows conditional expression of RNF5 in a transgenic mouse system.

FIG. 7A shows schematics depicting the transgenic constructs used to overexpress RNF5 in Mouse. The rtTA transcriptional activator is expressed under the control of a CMV enhancer/chicken β-actin promotor. The RNF5 transgene is expressed under the control of a tetracycline responsive promoter and is only activated in the presence of both the rtTA activator and doxycyclin. FIG. 7B shows tissue expression of RNF5 transgene in double transgenic animals. DTg animals (rtTA RNF5) were treated with 2 mg/ml of doxycyclin in the drinking water during 10 days and RNF5 protein levels were monitored in different organs. RNF5 expression was assessed using RNF5 polyclonal antibody, either by straight western (skeletal muscle) or after immunoprecipitation (Heart, Kidney). α-tubulin was used as a normalization control. Two different controls for the DTg expression were included: double transgenic litter mates not treated with doxycyclin and single transgenic animals litter mates (RNF5) treated with doxycyclin. FIG. 7C shows phenotypic comparison of a representative DTg animal overexpressing RNF5 after 4 weeks of doxycyclin treatment with its control littermate. FIG. 7D shows weight curve comparison of RNF5 overexpressing DTg animals and their matching control along the doxycyclin treatment. The body mass of individual animals was monitored every week during 6 weeks after the beginning of the treatment. Graphs represent the relative change in body mass relative to the original weight of a single animal (n=5). Note: In the same gender and age class, both experimental and control animals had the same external appearance and similar weight at the beginning of the treatment D. FIG. 7E shows representative X-Ray analysis of RNF5 overexpressing mouse and its matching control after 6 weeks of doxycyclin treatment.

FIG. 8 shows RNF5 overexpressing mice exhibit an altered muscle structure. FIG. 8A-C show HNE analysis of triceps brachii (A), vastus lateralis (B) and tibialis anterior (C) cross-section from RNF5 or rtTA RNF5 animals treated with doxycyclin for 6 weeks. Quantitation of fiber cross section area corresponding to each muscle type are shown on the bottom panel. FIG. 8D shows average muscle mass of RNF5 and rtTA RNF5 animals treated with doxycyclin for 6 weeks. Each muscle was extracted, trimmed under the microscope and weighted on a precision scale (n=5 for experimental and control group). FIG. 8E shows average organ mass of RNF5 and rtTA RNF5 animals treated with doxycyclin for 6 weeks. The same procedure was followed as in D.

FIG. 9 shows RNF5 overexpression is associated with high level of degeneration-coupled regeneration. Shown is the quantification of the number of regenerative myofibers, expressed as the average number of embMHC positive fibers (early regenerative fibers) and of centrally nucleated fibers.

FIG. 10 shows RNF5 transgene localizes to the ER and its overexpression correlates with altered ER function. Shown is the expression level of select ER stress marker in the muscles of DTg animals compared to their control littermate. Western blot analysis of muscle extract was probed with Grp78, PDI and Grp94 antibodies. RNF5 and GAPDH antibodies were used for RNF5 expression and loading controls.

### DETAILED DESCRIPTION OF THE INVENTION

The disclosed method and compositions may be understood more readily by reference to the following detailed description of particular embodiments and the Example included therein and to the Figures and their previous and following description.

### A. Definitions

It must be noted that as used herein and in the appended claims, the singular forms “a,” “an,” and “the” include plural reference unless the context clearly dictates otherwise. Thus, for example, reference to “a polypeptide” includes a plurality of such polypeptides, reference to “the polypeptide” is a reference to one or more polypeptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### B. RNF5

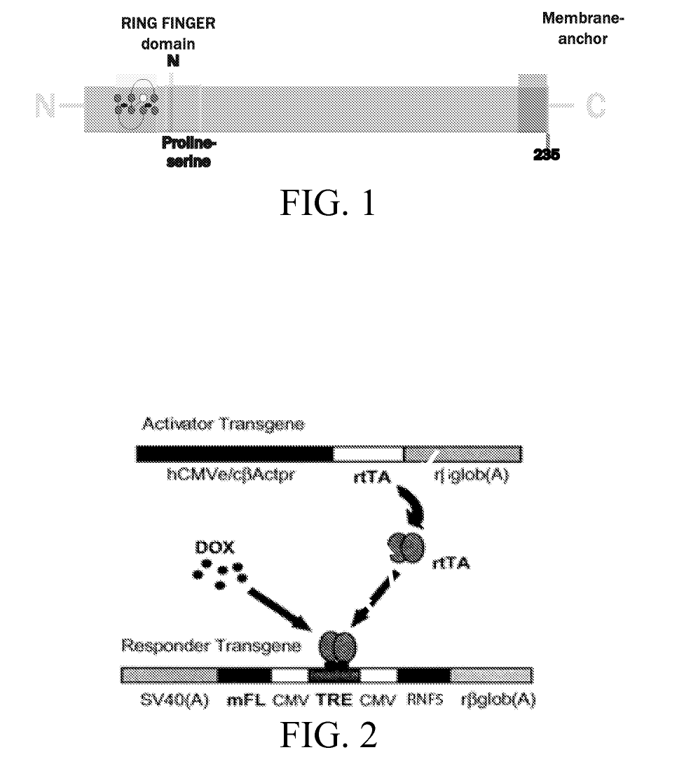

RNF5 is a RING domain containing protein also know as RMA-1 (Matsuda, N. et al. 1998; Matsuda, N., et al. 2001) that possesses E3 ubiquitin ligase activity. RNF5 was first identified in C. elegans (Kyushiki, H., et al. 1997) where it has also been characterized (Broday, L., et al. 2004). In addition to its RING domain, which is similar to those seen in other E3 ligases including cb1, Mdm2 and BRCA1, RNF5 comprises a hydrophobic C-terminal domain. This domain serves as a membrane anchoring subunit and a Formin Homology Domain (FIG. 1), which has been implicated in actin/cytoskeletal organization (Watanabe, N. et al. 2004). RNF5 expression is primarily seen within the leading edge of the cell, and is altered during adhesion, spreading and motility. RNF5 expression also resembles that of many of the cytoskeletal proteins that are contributing to cell motility and adhesion. Stainings that were performed with polyclonal antibodies indicate that RNF5 is primarily anchored to the ER membrane.

In C. elegans, RNF5 is expressed in the spermathecal septate junctions, germ cell membranes, and muscle dense bodies. In C. elegans, RNF5 regulates the localization and expression of Unc95 (Broday, L., et al. 2004), a LIM domain-containing protein whose deregulation/mutation is associated with uncoordinated movement (Zengel, J. M. et al. 1980) and altered muscle structure (Broday, L., et al. 2004). In vertebrates, RNF5 was found to associate with and ubiquitinate the LIM domain protein paxillin. RNF5-mediated ubiquitination of paxillin does not affect its stability. Instead it affects paxillin's subcellular localization by excluding it from focal adhesions, which results in impaired motility (Didier, C., et al. 2003).

Mice that lack RNF5 expression do not exhibit obvious phenotypes but are currently characterized for their ability to resist stress and exercise. However, as disclosed herein, overexpression of RNF5 in C. elegans results in >90% mortality within the L3 stage in development.

### C. Compositions

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a polypeptide is disclosed and discussed and a number of modifications that can be made to a number of molecules including the polypeptide are discussed, each and every combination and permutation of polypeptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

1. RNF5 Transgenic animals

Provided herein are non-human transgenic animals wherein nucleated cells of the animal comprise a nucleic acid encoding RNF5 operably linked to an expression control sequence. It is understood that expression of RNF5 encoded by the nucleic acid differs from native expression of RNF5 in the cells. Thus, in one aspect, the expression control sequence is not a naturally occurring RNF5 promoter and is therefore not operably linked to a nucleic acid encoding RNF5 in nature. As another example, the expression control sequence can be altered to alter expression of RNF5. The nucleated cells of the transgenic animals need not all have the nucleic acid and/or need not all exhibit expression of RNF5 that differs from native expression of RNF5. In some aspects, the RNF5 polypeptide has ubiquitin ligase activity. In some aspects, the nucleic acid encoding the RNF5 polypeptide is operably linked to an expression control sequence, wherein the RNF5 polypeptide is expressed at least in muscle cells.

The nucleic acid encoding RNF5 can be, for example, endogenous to the animal or exogenous to the animal. Exogenous to an animal or cell refers to a component that is not naturally present in the animal or cell, such as a component (a nucleic acid, for example) that is introduced into the animal or cell. Endogenous to an animal or cell refers to a component that is naturally present in the animal or cell, such as a component (a nucleic acid, for example) that is naturally present in the animal or cell and not artificially introduced into the animal or cell. The nucleic acid encoding RNF5 can be, for example, homologous to the animal or heterologous to the animal. Heterologous to an animal or cell refers to a type of component that does not naturally exist in the animal or cell, such as a type of component (a type of gene, for example) that comes from a different type of animal or cell. Homologous to an animal or cell refers to a type of component that naturally exists in the animal or cell, such as a type of component (a type of gene, for example) that comes from the same type of animal or cell and not from a different type of animal or cell. By way of illustration, a mouse insulin gene is homologous to any mouse or mouse cell and heterologous to, for example, cow, dog and human or a cow, dog or human cell. A mouse insulin gene artificially introduced into, for example, a cow, dog or human cell is exogenous to the cell (the mouse gene is also heterologous to the cell). Likewise, a mouse insulin gene artificially introduced into a mouse cell is exogenous to the mouse cell (it is nevertheless homologous to the mouse cell). The native nucleic acid encoding mouse insulin in a mouse cell is endogenous to the mouse cell and is endogenous to any other cell except cells descended from the mouse cell (the native nucleic acid encoding mouse insulin is also homologous to the mouse cell and any other mouse cell and heterologous to any non-mouse cell).

i. Animals

By a “transgene” is meant a nucleic acid sequence that is inserted by artifice into a cell and becomes a part of the genome of that cell and its progeny. Such a transgene may be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form or in a different chromosomal location. A transgene can include one or more transcriptional regulatory sequences and any other nucleic acid, such as introns, that may be useful or necessary for optimal expression of a selected nucleic acid. A transgene can be as few as a couple of nucleotides long, but is preferably at least about 50, 100, 150, 200, 250, 300, 350, 400, or 500 nucleotides long or even longer and can be, e.g., an entire genome. A transgene can be coding or non-coding sequences, or a combination thereof. A transgene usually comprises a regulatory element that is capable of driving the expression of one or more transgenes under appropriate conditions. By “transgenic animal” is meant an animal comprising a transgene as described above. Transgenic animals are made by techniques that are well known in the art. The disclosed nucleic acids, in whole or in part, in any combination, can be transgenes as disclosed herein.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein.

The disclosed transgenic animals can be any non-human animal, including a non-human mammal (e.g., mouse, rat, rabbit, squirrel, hamster, rabbits, guinea pigs, pigs, micro-pigs, prairie dogs, baboons, squirrel monkeys and chimpanzees, etc), bird or an amphibian, in which one or more cells contain heterologous nucleic acid introduced by way of human intervention, such as by transgenic techniques well known in the art. For example, the animal can be selected from the group consisting of avian, bovine, canine, caprine, equine, feline, leporine, murine, ovine, porcine, non-human primate. Thus, the animal can be a mouse, dog or cat.

Generally, the nucleic acid is introduced into the cell, directly or indirectly, by introduction into a precursor of the cell, such as by microinjection or by infection with a recombinant virus. The disclosed transgenic animals can also include the progeny of animals which had been directly manipulated or which were the original animal to receive one or more of the disclosed nucleic acids. This molecule may be integrated within a chromosome, or it may be extrachromosomally replicating DNA. For techniques related to the production of transgenic animals, see, inter alia, Hogan et al (1986) Manipulating the Mouse Embryo—A Laboratory Manual Cold Spring Harbor Laboratory, Cold Spring Harbor, N.Y., 1986).

Animals suitable for transgenic experiments can be obtained from standard commercial sources such as Charles River (Wilmington, Mass.), Taconic (Germantown, N.Y.), and Harlan Sprague Dawley (Indianapolis, Ind.). For example, if the transgenic animal is a mouse, many mouse strains are suitable, but C57BL/6 female mice can be used for embryo retrieval and transfer. C57BL/6 males can be used for mating and vasectomized C57BL/6 studs can be used to stimulate pseudopregnancy. Vasectomized mice and rats can be obtained from the supplier. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), the teachings of which are generally known and are incorporated herein.

Transgenic animals can be identified by analyzing their DNA. For this purpose, for example, when the transgenic animal is an animal with a tail, such as rodent, tail samples (1 to 2 cm) can be removed from three week old animals. DNA from these or other samples can then be prepared and analyzed, for example, by Southern blot, PCR, or slot blot to detect transgenic founder (F (0)) animals and their progeny (F (1) and F (2)). Thus, also provided are transgenic non-human animals that are progeny of crosses between a transgenic animal of the invention and a second animal. Transgenic animals can be bred with other transgenic animals, where the two transgenic animals were generated using different transgenes, to test the effect of one gene product on another gene product or to test the combined effects of two gene products.

ii. Phenotype

It has been discovered that transgenic expression of RNF5 can result in muscle wasting in the animal. Muscle wasting, also known as muscle atrophy, refers the wasting or loss of muscle tissue resulting from disease or lack of use. The majority of muscle atrophy in the general population results from disuse. People with sedentary jobs and senior citizens with decreased activity can lose muscle tone and develop significant atrophy. This type of atrophy is reversible with vigorous exercise. Bed-ridden people can undergo significant muscle wasting. Astronauts, free of the gravitational pull of Earth, can develop decreased muscle tone and loss of calcium from their bones following just a few days of weightlessness.

Muscle atrophy resulting from disease rather than disuse is generally one of two types, that resulting from damage to the nerves that supply the muscles, and disease of the muscle itself. Examples of diseases affecting the nerves that control muscles would be poliomyelitis, amyotrophic lateral sclerosis (ALS or Lou Gehrig's disease), and Guillain-Barre syndrome. Examples of diseases affecting primarily the muscles would include muscular dystrophy, myotonia congenita, and myotonic dystrophy as well as other congenital, inflammatory or metabolic myopathies.

Transgenic expression of RNF5 can result in a muscular dystrophy. Muscular dystrophy (MD), also known as inherited myopathy, refers to a group of disorders characterized by progressive muscle weakness and loss of muscle tissue. The group of diseases called muscular dystrophies (MD) includes many inherited disorders such as Becker's muscular dystrophy, Duchenne muscular dystrophy, facioscapulohumeral muscular dystrophy, limb-girdle muscular dystrophy, Emery-Dreifuss muscular dystrophy, myotonic dystrophy, and myotonia congenital. These disorders are distinguished from each other by the type of inheritance (sex-linked, dominant gene, recessive gene), the age when symptoms appear, and the types of symptoms that develop.

Symptoms vary with the different types of muscular dystrophy. Some types, such as Duchenne muscular dystrophy, are ultimately fatal while other types have associated muscle weakness but cause little disability and are associated with normal life expectancy.

The muscles primarily affected vary, but can be around the pelvis, shoulder, face or elsewhere. The age of onset can vary as well, with more severe subtypes tending to occur earlier in childhood. Examination and history help to distinguish the type of MD. Specific muscle groups are affected by different types of MD. Often, there is a loss of muscle mass (wasting), which may be disguised in some types of muscular dystrophy by an accumulation of fat and connective tissue that makes the muscle appear larger (pseudohypertrophy). Joint contractures are common. Shortening of the muscle fibers, fibrosis of the connective tissue and scarring slowly destroy muscle function. Some types of MD involve the heart muscle, causing cardiomyopathy or arrhythmias.

A muscle biopsy may be the primary test used to confirm the diagnosis. In some cases a DNA test from the blood may be sufficient. A serum CPK (an enzyme found in muscle) may be elevated. An EMG (electromyography) may confirm that weakness is caused by destruction of muscle tissue rather than damage to nerves. An ECG (electrocardiography) to monitor changes in cardiac status. This disease may also alter the results of myoglobin in urine/serum, LDH, creatinine, AST, aldolase.

iii. RNF5 Transgene

The nucleic acid encoding RNF5 can be heterologous to the animal. For example, the nucleic acid can encode human RNF5. Thus, the nucleic acid encoding RNF5 can comprise the nucleic acid sequence set forth in SEQ ID NO:1 or SEQ ID NO:3. The nucleic acid encoding RNF5 can also have at least about 70% or 75% or 80% or 85% or 90% or 95% sequence identity to the nucleic acid sequence set forth in SEQ ID NO:1 or SEQ ID NO:3, or a fragment thereof of at least 20, 30, 40, 50, 60, 70, 80, 90, 100, 110, 120, 130, 140, 150, 160, 170, 180, 190, 200, 250, 300, 350, 400, 450, or 500 nucleotides in length. The nucleic acid encoding RNF5 can also have at least about 70% or 75% or 80% or 85% or 90% or 95% sequence identity to the nucleic acid sequence set forth in SEQ ID NO:1 or SEQ ID NO:3, or a fragment thereof of at least 20, 30, 40, 50, 60, 70, 80, 90, 100, 110, 120, 130, 140, 150, 160, 170, 180, 190, 200, 250, 300, 350, 400, 450, or 500 nucleotides in length, wherein the polypeptide has ubiquitin ligase activity. The nucleic acid encoding RNF5 can also hybridize under stringent conditions to a hybridization probe consisting of the nucleic acid sequence set forth in SEQ ID NO:1 or SEQ ID NO:3. The nucleic acid encoding RNF5 can also hybridize under stringent conditions to a hybridization probe consisting of the nucleic acid sequence set forth in SEQ ID NO:1 or SEQ ID NO:3, wherein the polypeptide has ubiquitin ligase activity. The nucleic acid can encode a polypeptide having the sequence set forth in SEQ ID NO:2, or a fragment thereof of at least 20, 30, 40, 50, 60, 70, 80, 90, 100, 105, 110, 115, 120, 125, 130, 135, 140, 145, 150, 155, 160, 165, 170, or 175 amino acids in length. The nucleic acid can encode a polypeptide having the sequence set forth in SEQ ID NO:2, or a fragment thereof of at least 20, 30, 40, 50, 60, 70, 80, 90, 100, 105, 110, 115, 120, 125, 130, 135, 140, 145, 150, 155, 160, 165, 170, or 175 amino acids in length, wherein the polypeptide has ubiquitin ligase activity. The nucleic acid can encode a polypeptide having at least about 70% or 75% or 80% or 85% or 90% or 95% sequence identity to the amino acid sequence set forth in SEQ ID NO:2, or a fragment thereof of at least 20, 30, 40, 50, 60, 70, 80, 90, 100, 105, 110, 115, 120, 125, 130, 135, 140, 145, 150, 155, 160, 165, 170, or 175 amino acids in length. The nucleic acid can encode a polypeptide having at least about 70% or 75% or 80% or 85% or 90% or 95% sequence identity to the amino acid sequence set forth in SEQ ID NO:2, or a fragment thereof of at least 20, 30, 40, 50, 60, 70, 80, 90, 100, 105, 110, 115, 120, 125, 130, 135, 140, 145, 150, 155, 160, 165, 170, or 175 amino acids in length, wherein the polypeptide has ubiquitin ligase activity. The nucleic acid encoding RNF5 can be at least 20, 30, 40, 50, 60, 70, 80, 90, 100, 110, 120, 130, 140, 150, 160, 170, 180, 190, 200, 250, 300, 350, 400, 450, or 500 nucleotides in length. The nucleic acid encoding RNF5 can be at least 20, 30, 40, 50, 60, 70, 80, 90, 100, 110, 120, 130, 140, 150, 160, 170, 180, 190, 200, 250, 300, 350, 400, 450, or 500 nucleotides in length, wherein the polypeptide has ubiquitin ligase activity.

iv. Expression Control Sequence

Nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

The nucleic acid encoding the expression control sequence can be heterologous to the animal. The expression control sequence can comprise a constitutive promoter. The expression control sequence can comprise a cell-specific promoter. The expression control sequence can comprise a muscle-specific promoter. For example, the cell-specific promoter can be muscle creatine kinase (MCK) promoter (Fabre et al. J Gene Med. 2006 8(5):636-45), desmin promoter (Raats et al. Eur J. Cell Biol. 1996 71(3):221-36), or myoglobin promoter.

The expression control sequence can comprise an inducible promoter. Alternatively, the nucleated cells of the provided animal can further comprise a transgene encoding a transactivator protein, wherein the transactivator protein conditionally induces expression of the transgene encoding RNF5. For example, inducible expression by the transactivator protein can be conditioned on the presence of tetracycline or derivative thereof. Likewise, inducible expression by the transactivator protein can conditioned on the absence of tetracycline or derivative thereof. An example of such an inducible system is diagramed in FIG. 2. Numerous other control sequences and systems are known and can be used with the disclosed transgenes and transgenic animals.

a. Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers. The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b. Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

2. Modulator of RNF5

Provided herein are modulators of RNF5 levels or activity. “Activity,” as used herein, refers to any function or process of a composition disclosed herein and includes, for example, transcription, translation, post-translational modification, translocation, homophilic or heterophilic binding, secretion, endocytosis, or degradation. Disclosed therefore are compositions that inhibit one or more activities of RNF5.

i. Gene Knockout

RNF5 levels can be modulated in a non-human animal by deletion of the RNF5 gene by homologous recombination. The resulting heterozygous or homozygous null RNF5 KO mice can also be used in the herein disclosed methods.

ii. Knockdown of Gene Expression

RNF5 levels can be modulated at the gene expression level. Thus, the modulator of RNF5 can be a functional nucleic acid such as a gene expression inhibitor. Methods of targeting gene expression are generally based on the sequence of the gene to be targeted. Disclosed are any such methods that can be applied to the targeted knockdown of RNF5. By “knockdown” is meant a decrease in detectable mRNA expression. Nucleic acids are generally used to knockdown gene expression and generally comprise a sequence capable of hybridizing to the target sequence, such as mRNA. Examples of such functional nucleic acids include antisense molecules, ribozymes, triplex forming nucleic acids, external guide sequences (EGS), and small interfering RNAs (siRNA).

Antisense molecules are designed to interact with a target nucleic acid molecules through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437. However, the effect of iRNA or siRNA or their use is not limited to any type of mechanism.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Disclosed herein are any ribozymes designed as described above based on the sequences for RNF5. Hammerhead ribozymes can cleave RNA substrates at for example, a 5′-GUC-3′ sequence, cleaving the mRNA immediately 3′ to the GUC site. Engineered hammerhead ribozymes, which cleave at a different sequence are known and disclosed, for example, in the patents disclosed herein, and are incorporated by reference. A hammerhead ribozyme is typically composed of three parts. The first part is a region which will hybridize to the sequence 5′ of the GUC recognition site, and can be called a first hybridization arm. A second part consists of a core catalytic domain of the hammerhead ribozyme. A third part consists of sequence capable of hybridizing to the sequence immediately 3′ to the GUC cleavage site, and can be called a second hybridization arm. The hybridization arms can be any length allowing binding to the substrate, such as, from 3-40 nucleotides long, 5-30 nucleotides long, 8-20, nucleotides long and 10-15 nucleotides long, as well as any length up to 50 nucleotides. As an example, hammerhead ribozymes can be designed by identifying a nucleic acid triplet GUC within the mRNA target sequence, and then identifying the appropriate hybridizing arms as discussed herein to the catalytic core as discussed herein. Furthermore, using assays as discussed herein, one can test a given ribozyme (or any functional nucleic acid, such as an siRNA or antisense) for its level of activity, both in vitro and in vivo.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)). Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules are found in the following non-limiting list of U.S. Pat. Nos. 5,168,053, 5,624,824, 5,683,873, 5,728,521, 5,869,248, and 5,877,162.

Gene expression can also be effectively silenced in a highly specific manner through RNA interference (RNAi). This silencing was originally observed with the addition of double stranded RNA (dsRNA) (Fire, A., et al. (1998) Nature, 391, 806 811) (Napoli, C., et al. (1990) Plant Cell 2, 279 289) (Hannon, G. J. (2002) Nature, 418, 244 251). Once dsRNA enters a cell, it is cleaved by an RNase III like enzyme, Dicer, into double stranded small interfering RNAs (siRNA) 21-23 nucleotides in length that contains 2 nucleotide overhangs on the 3′ ends (Elbashir, S. M., et al. (2001) Genes Dev., 15:188-200) (Bernstein, E., et al. (2001) Nature, 409, 363 366) (Hammond, S. M., et al. (2000) Nature, 404:293-296). In an ATP dependent step, the siRNAs become integrated into a multi-subunit protein complex, commonly known as the RNAi induced silencing complex (RISC), which guides the siRNAs to the target RNA sequence (Nykanen, A., et al. (2001) Cell, 107:309 321). At some point the siRNA duplex unwinds, and it appears that the antisense strand remains bound to RISC and directs degradation of the complementary mRNA sequence by a combination of endo and exonucleases (Martinez, J., et al. (2002) Cell, 110:563-574). However, the effect of iRNA or siRNA or their use is not limited to anytype of mechanism.

Short Interfering RNA (siRNA) is a double-stranded RNA that can induce sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing can be achieved in mammalian cells using synthetic, short double-stranded RNAs that mimic the siRNAs produced by the enzyme dicer (Elbashir, S. M., et al. (2001) Nature, 411:494 498) (Ui-Tei, K., et al. (2000) FEBS Lett 479:79-82). siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, The Netherlands). siRNA can also be synthesized in vitro using kits such as Ambion's SILENCER siRNA Construction Kit. Disclosed herein are any siRNA designed as described above based on the sequences for the herein disclosed inflammatory mediators.

The production of siRNA from a vector is more commonly done through the transcription of a shRNA. Kits for the production of vectors comprising shRNA are available, such as for example Imgenex's GeneSuppressor Construction Kits and Invitrogen's BLOCK-iT inducible RNAi plasmid and lentivirus vectors. Disclosed herein are any shRNA designed as described above based on the sequences for RNF5. Examples of shRNA that can be used to inhibit RNF5 expression are set forth in SEQ ID NOs:11, 12 and 13.

iii. Inhibition of Binding

Another activity of an RNF5 that can be targeted is homophilic and heterophilic binding to other molecules, such as, for example, receptors. Thus, the RNF5 inhibitor can be a ligand binding inhibitor. Methods for inhibiting the binding of a protein to its receptor can, for example, be based on the use of molecules that compete for the binding site of either the ligand or the receptor.

Thus, a ligand binding inhibitor can be, for example, a polypeptide that competes for the binding of a receptor without activating the receptor. Likewise, a ligand binding inhibitor can be a decoy receptor that competes for the binding of ligand. Such a decoy receptor can be a soluble receptor (e.g., lacking transmembrane domain) or it can be a mutant receptor expressed in a cell but lacking the ability to transduce a signal (e.g., lacking cytoplasmic tail). Antibodies specific for either a ligand or a receptor can also be used to inhibit binding. The ligand binding inhibitor can also be naturally produced by a subject. Alternatively, the inhibitory molecule can be designed based on targeted mutations of either the receptor or the ligand. Thus, as an illustrative example, the ligand binding inhibitor can be paxillin or a fragment thereof comprising the RNF5 binding site. For example, paxillin binds RNF5 at its amino-terminus. Thus the ligand binding inhibitor can be an amino terminal fragment of paxillin lacking the LIM domains.

The ligand binding inhibitor can also be Salmonella type III effector SopA or a fragment thereof comprising the RNF5 binding site. The ligand binding inhibitor can also be a mutant cystic fibrosis transmembrane conductance regulator (CFTR), such as CFTRΔF508, or a fragment thereof comprising the RNF5 binding site. The ligand binding inhibitor can also be TCRα or a fragment thereof comprising the RNF5 binding site. The ligand binding inhibitor can also be CD3δ or a fragment thereof comprising the RNF5 binding site. The ligand binding inhibitor can also be Unc95 or a fragment thereof comprising the RNF5 binding site.

As used herein, a ligand binding inhibitor can also be a decoy molecule that competes for the binding of RNF5-associated proteins, including co-factors. For example, proteins with similar structure to RNF5, namely RNF185, can associate and regulate RNF5 activity. Thus, the ligand binding inhibitor can be RNF185, or a fragment thereof comprising the RNF5 binding site. Thus, the nucleic acid encoding RNF185 can comprise the nucleic acid sequence set forth in SEQ ID NO:4. The nucleic acid encoding RNF185 can also have at least about 70% or 75% or 80% or 85% or 90% or 95% sequence identity to the nucleic acid sequence set forth in SEQ ID NO:4, or a fragment thereof of at least 20, 30, 40, 50, 60, 70, 80, 90, 100, 110, 120, 130, 140, 150, 160, 170, 180, 190, 200, 250, 300, 350, 400, 450, or 500 nucleotides in length. The nucleic acid encoding RNF185 can also hybridizes under stringent conditions to a hybridization probe consisting of the nucleic acid sequence set forth in SEQ ID NO:4. The nucleic acid can encode a polypeptide having the sequence set forth in SEQ ID NO:5. The nucleic acid can encode a polypeptide having at least about 70% or 75% or 80% or 85% or 90% or 95% sequence identity to the amino acid sequence set forth in SEQ ID NO:5, or a fragment thereof of at least 20, 30, 40, 50, 60, 70, 80, 90, 100, 105, 110, 115, 120, 125, 130, 135, 140, 145, 150, 155, 160, 165, 170, or 175 amino acids in length. The nucleic acid encoding RNF185 can be at least 20, 30, 40, 50, 60, 70, 80, 90, 100, 110, 120, 130, 140, 150, 160, 170, 180, 190, 200, 250, 300, 350, 400, 450, or 500 nucleotides in length.

Examples of RNF5 associated proteins are set forth in Table 1. Other such proteins can be identified by routine methods known in the art.

TABLE 1
RNF5 associated proteins identified in mass spec analysis*

| Protein name | GI number | Accession No. |
|---|---|---|
| fatty acid synthase | Gi 21618359 | NP_004095.3 |
| valosin-containing protein | Gi 6005942 | NP_009057.1 |
| vesicle transport related protein | Gi 7706407 | NO_057247.1 |
| Ribophorin | Gi 4506675 | NP_002941 |
| protein disulfide isomerase related protein | Gi 5031973 | NP_005733.1 |
| transmembrane protein (63 kd), | Gi 19920317 | |
| endoplasmic reticulum/golgi inter | | |
| prostate apoptosis response protein 4. | Gi 4505613 | NP_002574.1 |
| SPFH2 | Gi 45709604 | AAH67765 |
| c8 orf2 SPFH family member | Gi 37181322 | AAQ88475 |
| KIAA0917 protein | Gi 34327968 | BAA74940.2 |
| KIAA0090 protein | Gi 71891723 | BAA07645.2 |
| heat shock 70 kda protein 8 isoform 1 | Gi 5729877 | NP_006588.1 |
| SPFH domain family member 1 | Gi 21618849 | AAH31791.1 |
| DNJBC - Dna homologue subfamily B | Gi 44889076 | Q9NXW2 |
| member 12. | | |
| BRI3 binding protein | Gi 21961229 | AAH34525.1 |
| BiP/grp78 | Gi 1143492 | CAA61201.1 |
| family with sequence similarity 62 | Gi 13436458 | AAH04998 |
| (C2 domain containing). | | |
| DEAH box polypeptide 30 isoform 1 | Gi 20336294 | NP_619520.1 |
| skeletal muscle chloride channel protein. | Gi 6006527 | CAB56792.1 |
| chromosome 8 open reading frame 2 | Gi 6005721 | NP_009106 |

*data represents 2 analyses performed on total proteins and ER residing proteins which associate with RNF5.

3. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

4. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein. It is understood that those of skill in the art understand that if a composition or method meets any one of these criteria for determining hybridization either collectively or singly it is a composition or method that is disclosed herein.

5. Nucleic Acids

There are a variety of molecules disclosed herein that are nucleic acid based, including for example the nucleic acids that encode, for example, RNF5 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

i. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

ii. Sequences

There are a variety of sequences related to, for example, RNF5 as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

iii. Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

6. Proteins and Peptides

As discussed herein there are numerous variants of the RNF5 protein that are known and herein contemplated. There are also derivatives of RNF5 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 2
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 3
Amino Acid Substitutions

| Original | Exemplary Conservative Substitutions, |
|---|---|
| Residue | others are known in the art. |
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 3, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:2 sets forth a particular sequence of RNF5 protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

For example, one of the many nucleic acid sequences that can encode the protein sequence set forth in SEQ ID NO:2 is set forth in SEQ ID NO:1. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 2 and Table 3. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂—CH₂—CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### D. Methods of Using the Compositions

Also provided herein is a method of producing a non-human transgenic animal comprising introducing a nucleotide sequence encoding RNF5 operably linked to an expression control sequence into a fertilized animal oocyte; allowing the fertilized animal oocyte to develop to term; and identifying a transgenic animal whose genome comprises the RNF5 nucleotide sequence, wherein expression of the RNF5 results in muscle wasting in the animal. Also provided herein is a method for producing a non-human transgenic animal comprising providing a vector comprising a nucleotide sequence encoding RNF5 operably linked to an expression control sequence; introducing the expression vector into a fertilized animal oocyte; allowing said fertilized animal oocyte to develop to term; and identifying a transgenic animal whose genome comprises the RNF5 nucleotide sequence, wherein expression of said RNF5 results in muscle wasting in the animal.

Also provided herein is a method comprising administering a vector comprising a nucleotide sequence encoding RNF5 operably linked to an expression control sequence to an animal, wherein expression of said RNF5 in the muscle results in muscle wasting in the animal. Also provided herein is a method comprising providing a vector comprising a nucleotide sequence encoding RNF5 operably linked to an expression control sequence; administering the expression vector to an animal, wherein expression of said RNF5 in the muscle results in muscle wasting in the animal.

Also provided herein is a transgenic non-human animal having a phenotype characterized by altered expression of RNF5 polypeptide, the phenotype being conferred by a transgene contained in the cells of the animal, the transgene comprising a nucleic acid sequence which encodes an RNF5 polypeptide. In some aspects, the RNF5 polypeptide can have ubiquitin ligase activity. In some aspects, the nucleic acid encoding the RNF5 polypeptide can be operably linked to an expression control sequence, wherein the RNF5 polypeptide is expressed at least in muscle cells. The cells containing the transgene can be somatic cells. The cells containing the transgene can be germ cells.

Also provided is a method of treating or preventing muscle wasting in a subject, wherein the subject has elevated levels of RNF5 in said muscle, comprising administering to the subject a modulator of RNF5. The modulator of RNF5 can be a functional nucleic acid.

In one aspect of the provided method, the subject has been diagnosed with muscular dystrophy.

Also provided is a method for diagnosing a muscle wasting disease in a subject, comprising acquiring a sample from the muscle of said subject, detecting RNF5 levels or activity in the sample, wherein high levels or activity of RNF5 as compared to a control indicates muscle wasting in the subject.

Also provided is a method of identifying a subject at risk for muscle wasting disease, comprising detecting RNF5 levels or activity in a sample from the muscle of said subject, wherein high levels or activity of RNF5 as compared to a control indicates a risk for muscle wasting in the subject. Also provided is a method of identifying a subject at risk for muscle wasting disease, comprising acquiring a sample from the muscle of said subject, detecting RNF5 levels or activity in the sample, wherein high levels or activity of RNF5 as compared to a control indicates a risk for muscle wasting in the subject.

Also provided is a method of assessing the severity of muscle wasting in a subject, comprising detecting RNF5 levels or activity in a sample from the muscle of said subject, wherein the levels or activity of RNF5 as compared to a control correlate with the severity of muscle wasting in the subject. Also provided is a method of assessing the severity of muscle wasting in a subject, comprising acquiring a sample from the muscle of said subject, detecting RNF5 levels or activity in the sample, wherein the levels or activity of RNF5 as compared to a control correlate with the severity of muscle wasting in the subject.

Also provided is a method of identifying targets of RNF5, comprising detecting binding of a candidate substrate to RNF5 in a sample comprising RNF5 as compared to a control lacking RNF5, or detecting degradation of the candidate substrate in a sample comprising RNF5 as compared to a control lacking RNF5, wherein detectable binding of the candidate substrate to RNF5 or detectable degradation of the candidate substrate in the presence of RNF5 indicates that the candidate substrate is a target of RNF5.

1. Method of Screening

Also provided is a method of screening for an agent for use in treating or preventing muscle wasting, comprising administering a candidate agent to a sample comprising RNF5, monitoring the sample for changes in the expression of RNF5, RNF5 ligase activity, degradation of an RNF5 substrate, or binding of RNF5 to a substrate, wherein a detectable change in any one or more of these activities an indication that the candidate can be used for treating or preventing muscle wasting. For example, a decrease in the expression of RNF5, RNF5 ligase activity, degradation of an RNF5 substrate, or binding of RNF5 to a substrate can indicate that the candidate can be used for treating or preventing muscle wasting. For example, an increase in degradation of an RNF5 substrate or binding of RNF5 to a substrate can indicate that the candidate can be used for treating or preventing muscle wasting. RNF5 can be expressed in said sample by a cell comprising a nucleic acid encoding RNF5 operably linked to an expression control sequence. Methods for detecting gene expression, ligase activity, protein degradation, and substrate binding are known in the art, and any such detection method is contemplated for use herein.

The sample comprising RNF5 can be a non-human transgenic animal wherein nucleated cells of the animal comprise a nucleic acid encoding RNF5 operably linked to an expression control sequence, wherein expression of the RNF5 encoded by the nucleic acid differs from native expression of RNF5 in the cells.

Also provided is a method of making an agent that modulates RNF5 activity, comprising administering a candidate agent to a sample comprising RNF5, monitoring the sample for changes in the expression of RNF5, RNF5 ligase activity, degradation of an RNF5 substrate, or binding of RNF5 to a substrate, wherein a detectable change in any one or more of these activities indicates that the candidate agent modulates RNF5 activity, making the candidate agent.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein.

Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods.

Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity of RNF5 should be employed whenever possible. Candidate agents can also be natural targets or binding partners of RNF5, or derivatives of natural targets and binding partners of RNF5,

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that stimulates or inhibits RNF5. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art. Compounds identified as being of therapeutic value may be subsequently analyzed using animal models for diseases or conditions in which it is desirable to regulate or mimic activity of RNF5.

### E. Methods of Making the Compositions

The compositions disclosed herein and the compositions necessary to perform the disclosed methods can be made using any method known to those of skill in the art for that particular reagent or compound unless otherwise specifically noted.

1. Transgenic Mice Models

i. Methods of Producing Transgenic Animals

The nucleic acids and vectors provided herein can be used to produce transgenic animals. Various methods are known for producing a transgenic animal. In one method, an embryo at the pronuclear stage (a “one cell embryo”) is harvested from a female and the transgene is microinjected into the embryo, in which case the transgene will be chromosomally integrated into the germ cells and somatic cells of the resulting mature animal. In another method, embryonic stem cells are isolated and the transgene is incorporated into the stem cells by electroporation, plasmid transfection or microinjection; the stem cells are then reintroduced into the embryo, where they colonize and contribute to the germ line. Methods for microinjection of polynucleotides into mammalian species are described, for example, in U.S. Pat. No. 4,873,191, which is incorporated herein by reference. In yet another method, embryonic cells are infected with a retrovirus containing the transgene, whereby the germ cells of the embryo have the transgene chromosomally integrated therein. When the animals to be made transgenic are avian, microinjection into the pronucleus of the fertilized egg is problematic because avian fertilized ova generally go through cell division for the first twenty hours in the oviduct and, therefore, the pronucleus is inaccessible. Thus, the retrovirus infection method is preferred for making transgenic avian species (see U.S. Pat. No. 5,162,215, which is incorporated herein by reference). If microinjection is to be used with avian species, however, the embryo can be obtained from a sacrificed hen approximately 2.5 hours after the laying of the previous laid egg, the transgene is microinjected into the cytoplasm of the germinal disc and the embryo is cultured in a host shell until maturity (Love et al., Biotechnology 12, 1994). When the animals to be made transgenic are bovine or porcine, microinjection can be hampered by the opacity of the ova, thereby making the nuclei difficult to identify by traditional differential interference-contrast microscopy. To overcome this problem, the ova first can be centrifuged to segregate the pronuclei for better visualization.

The transgene can be introduced into embryonal target cells at various developmental stages, and different methods are selected depending on the stage of development of the embryonal target cell. The zygote is the best target for microinjection. The use of zygotes as a target for gene transfer has a major advantage in that the injected DNA can incorporate into the host gene before the first cleavage (Brinster et al., Proc. Natl. Acad. Sci., USA 82:4438-4442, 1985). As a consequence, all cells of the transgenic non-human animal carry the incorporated transgene, thus contributing to efficient transmission of the transgene to offspring of the founder, since 50% of the germ cells will harbor the transgene.

A transgenic animal can be produced by crossbreeding two chimeric animals, each of which includes exogenous genetic material within cells used in reproduction. Twenty-five percent of the resulting offspring will be transgenic animals that are homozygous for the exogenous genetic material, 50% of the resulting animals will be heterozygous, and the remaining 25% will lack the exogenous genetic material and have a wild type phenotype.

In the microinjection method, the transgene is digested and purified free from any vector DNA, for example, by gel electrophoresis. The transgene can include an operatively associated promoter, which interacts with cellular proteins involved in transcription, and provides for constitutive expression, tissue specific expression, developmental stage specific expression, or the like. Such promoters include those from cytomegalovirus (CMV), Moloney leukemia virus (MLV), and herpes virus, as well as those from the genes encoding metallothionein, skeletal actin, Phosphenolpyruvate carboxylase (PEPCK), phosphoglycerate (PGK), dihydrofolate reductase (DHFR), and thymidine kinase (TK). Promoters from viral long terminal repeats (LTRs) such as Rous sarcoma virus LTR also can be employed. When the animals to be made transgenic are avian, preferred promoters include those for the chicken [bgr]-globin gene, chicken lysozyme gene, and avian leukosis virus. Constructs useful in plasmid transfection of embryonic stem cells will employ additional regulatory elements, including, for example, enhancer elements to stimulate transcription, splice acceptors, termination and polyadenylation signals, ribosome binding sites to permit translation, and the like.

In the retroviral infection method, the developing non-human embryo can be cultured in vitro to the blastocyst stage. During this time, the blastomeres can be targets for retroviral infection (Jaenich, Proc. Natl. Acad. Sci. USA 73:1260-1264, 1976). Efficient infection of the blastomeres is obtained by enzymatic treatment to remove the zona pellucida (Hogan et al., Manipulating the Mouse Embryo (Cold Spring Harbor Laboratory Press, 1986). The viral vector system used to introduce the transgene is typically a replication-defective retrovirus carrying the transgene (Jahner et al., Proc. Natl. Acad. Sci., USA 82:6927-6931, 1985; Van der Putten et al., Proc. Natl. Acad. Sci. USA 82:6148-6152, 1985). Transfection is easily and efficiently obtained by culturing the blastomeres on a monolayer of virus producing cells (Van der Putten et al., supra, 1985; Stewart et al., EMBO J. 6:383-388, 1987). Alternatively, infection can be performed at a later stage. Virus or virus-producing cells can be injected into the blastocoele (Jahner et al., Nature 298:623-628, 1982). Most of the founders will be mosaic for the transgene since incorporation occurs only in a subset of the cells which formed the transgenic nonhuman animal. Further, the founder can contain various retroviral insertions of the transgene at different positions in the genome, which generally will segregate in the offspring. In addition, it is also possible to introduce transgenes into the germ line, albeit with low efficiency, by intrauterine retroviral infection of the mid-gestation embryo (Jahner et al., supra, 1982).

Embryonal stem cell (ES) also can be targeted for introduction of the transgene. ES cells are obtained from pre-implantation embryos cultured in vitro and fused with embryos (Evans et al. Nature 292:154-156, 1981; Bradley et al., Nature 309:255-258, 1984; Gossler et al., Proc. Natl. Acad. Sci., USA 83:9065-9069, 1986; Robertson et al., Nature 322:445-448, 1986). Transgenes can be efficiently introduced into the ES cells by DNA transfection or by retrovirus mediated transduction. Such transformed ES cells can thereafter be combined with blastocysts from a nonhuman animal. The ES cells thereafter colonize the embryo and contribute to the germ line of the resulting chimeric animal (see Jaenisch, Science 240:1468-1474, 1988).

“Founder” generally refers to a first transgenic animal, which has been obtained from any of a variety of methods, e.g., pronuclei injection. An “inbred animal line” is intended to refer to animals which are genetically identical at all endogenous loci.

ii. Crosses

It is understood that the animals provided herein can be crossed with other animals. For example, wherein the provided animals are mice, they can be crossed with Alzheimer's Mice to study the effects of inflammatory mediators, e.g. IL-1β, on Alzheimer's disease. The association between Aβ deposition and inflammatory changes is reinforced by studies of transgenic mice harboring familial AD mutant genes. In transgenic mice expressing the Swedish APP mutation (Tg2576, APP_{K670N,M67IL}; hereafter referred to as APPsw), microglial activation is intimately related to amyloid plaque deposition, with measures of both microglial size and activated microglial density being highest in the immediate vicinity of Aβ deposits [Frautschy, S. A, et al. Am. J. Pathol. (1998) 152:307-317]. These mice accumulate Aβ deposits over a protracted period of time, with plaques and glial changes becoming prominent after one year of age [Hsiao, K., P. Chapman, S, Nilsen, C. Eckman, Y. Harigaya, S. Younkin, F. Yang and G. Cole. Science (1996) 274:99-102]. Although other AD mouse models are available, the APPsw mice have been extensively characterized and offer an excellent resource for investigating mechanisms involved in Aβ deposition or Aβ induced inflammatory changes.

Other dystrophic transgenic animals can be crossed with the provided transgenic animals. Many mutant animal models of muscular dystrophy share common genetic and protein abnormalities similar to those of the human disease. The best example is a model of Duchenne muscular dystrophy (DMD), the mdx mouse (Collins et al. Int J Exp Pathol. 2003 84(4):165-72; De Luca et al. Neuromuscul Disord. 2002 12 Suppl 1:S142-6). Similar to dystrophic muscle in DMD patients, dystrophin protein is not expressed along the surface membrane, even though the mdx mouse has no apparent signs of muscular dysfunction. Because clinical and pathologic findings in the dystrophic (mxd) dog are similar to those in DMD patients, it also has been regarded as a good model for therapeutic trials. The best known and most extensively studied dy+dy+ mouse lacks merosin (laminin alpha2), which is one subunit of a basement membrane protein, laminin. Because approximately half of all patients with the classical form of congenital muscular dystrophy also lack merosin, availability of this animal has revived interest in the study of the pathologic mechanism of fiber necrosis resulting from this membrane defect. The dystrophic hamster is a model of limb-girdle muscular dystrophy with sarcoglycan deficiency in which one of the dystrophin-associated glycoproteins, delta-sarcoglycan, is defective. Because these animal models have common protein and genetic defects similar to those seen in people with muscular dystrophies, they have been widely used to examine the effectiveness of gene therapy and the administration of pharmacologic and trophic factors. Other examples of dystrophic animals include those with altered expression of Fukutin (Taniguchi et al. Hum Mol. Genet. 2006 15(8):1279-89) or Nesprin-2 (Zhang et al. J Cell Sci. 2005 118(Pt 4):673-87).

2. Delivery of the Compositions to Cells

Animal models of muscle wasting can also be produce by exogenous delivery of RNF5 or nucleic acids encoding RNF directly to the muscle. Thus, also provided herein are compositions and methods for the delivery of a nucleic acid encoding RNF5 to a muscle cell. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

i. Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Canter Res. 53:83-88, (1993)). As used herein, plasmid or viral vectors are agents that transport the disclosed nucleic acids, such as the nucleic acids encoding an inflammation molecule into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

a. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

b. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

c. Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

d. Lentiviral Vectors

The vectors can be lentiviral vectors, including but not limited to, SW vectors, HIV vectors or a hybrid construct of these vectors, including viruses with the HIV backbone. These vectors also include first, second and third generation lentiviruses. Third generation lentiviruses have lentiviral packaging genes split into at least 3 independent plasmids or constructs. Also vectors can be any viral family that share the properties of these viruses which make them suitable for use as vectors. Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses. Typically, a lentiviral vector contains the 5′ and 3′ LTR regions of a lentivirus, such as SW and HIV. Lentiviral vectors also typically contain the Rev Responsive Element (RRE) of a lentivirus, such as SW and HIV.

(A) Feline Immunodeficiency Viral Vectors

One type of vector that the disclosed constructs can be delivered in is the VSV-G pseudotyped Feline Immunodeficiency Virus system developed by Poeschla et al. Nature Med. (1998) 4:354-357 (Incorporated by reference herein at least for material related to FIV vectors and their use). This lentivirus has been shown to efficiently infect dividing, growth arrested as well as post-mitotic cells. Furthermore, due to its lentiviral properties, it allows for incorporation of the transgene into the host's genome, leading to stable gene expression. This is a 3-vector system, whereby each confers distinct instructions: the FIV vector carries the transgene of interest and lentiviral apparatus with mutated packaging and envelope genes. A vesicular stomatitis virus G-glycoprotein vector (VSV-G; Burns et al., Proc. Natl. Acad. Sci. USA 90:8033-8037. 1993) contributes to the formation of the viral envelope in trans. The third vector confers packaging instructions in trans (Poeschla et al. Nature Med. (1998) 4:354-357). FIV production is accomplished in vitro following co-transfection of the aforementioned vectors into 293-T cells. The FIV-rich supernatant is then collected, filtered and can be used directly or following concentration by centrifugation. Titers routinely range between 10⁴-10⁷ bfu/ml.

e. Packaging Vectors

As discussed above, retroviral vectors are based on retroviruses which contain a number of different sequence elements that control things as diverse as integration of the virus, replication of the integrated virus, replication of un-integrated virus, cellular invasion, and packaging of the virus into infectious particles. While the vectors in theory could contain all of their necessary elements, as well as an exogenous gene element (if the exogenous gene element is small enough) typically many of the necessary elements are removed. Since all of the packaging and replication components have been removed from the typical retroviral, including lentiviral, vectors which will be used within a subject, the vectors need to be packaged into the initial infectious particle through the use of packaging vectors and packaging cell lines. Typically retroviral vectors have been engineered so that the myriad functions of the retrovirus are separated onto at least two vectors, a packaging vector and a delivery vector. This type of system then requires the presence of all of the vectors providing all of the elements in the same cell before an infectious particle can be produced. The packaging vector typically carries the structural and replication genes derived from the retrovirus, and the delivery vector is the vector that carries the exogenous gene element that is preferably expressed in the target cell. These types of systems can split the packaging functions of the packaging vector into multiple vectors, e.g., third-generation lentivirus systems. Dull, T. et al., “A Third-generation lentivirus vector with a conditional packaging system” J. Virol 72(11):8463-71 (1998)

Retroviruses typically contain an envelope protein (env). The Env protein is in essence the protein which surrounds the nucleic acid cargo. Furthermore cellular infection specificity is based on the particular Env protein associated with a typical retrovirus. In typical packaging vector/delivery vector systems, the Env protein is expressed from a separate vector than for example the protease (pro) or integrase (in) proteins.

f. Packaging Cell Lines

The vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. One type of packaging cell line is a 293 cell line.

g. Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

ii. Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed nucleic acids or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

iii. In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

3. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

4. Peptide Synthesis

One method of producing the disclosed proteins, such as SEQ ID NO:2, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

5. Process for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO:1 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO:1, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO:1 and a sequence controlling the expression of the nucleic acid.

Disclosed are nucleic acid molecules produced by the process comprising linking in an operative way a nucleic acid molecule comprising a sequence encoding a peptide set forth in SEQ ID NO:2 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO:2 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO:2, wherein any change is a conservative change, and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### F. Examples

### 1. Example 1

### RNF5 Transgenic Mice

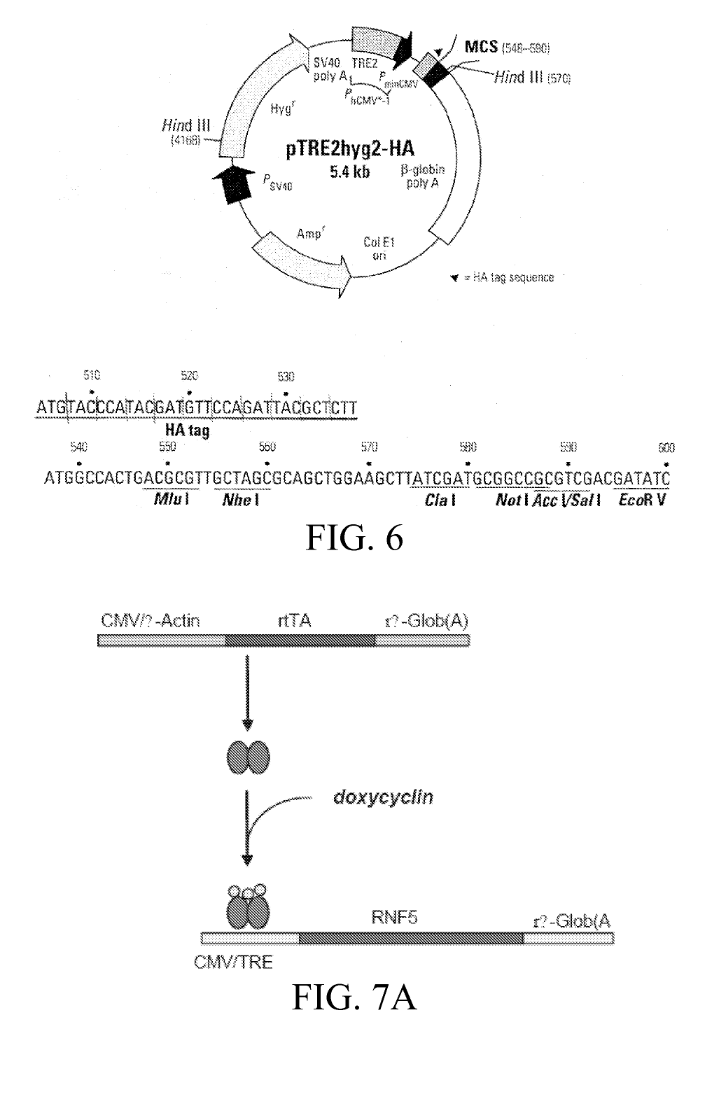

Tetracycline (tet) inducible transgenic RNF5 mice under the control of beta actin promoter were developed (FIG. 2). FIG. 6 shows the pTRE2hyg2-HA construct used to generate the RNF-5 transgenic mice. This construct was previously shown to express in number of tissues, including heart, liver, kidney, skin, brain and skeletal muscle (Manfra, D. J., et al. 2003). Table 4 discloses the features of the pTRE2hyg2-HA construct.

TABLE 4
pTRE2hyg2-HA feature

| Feature | Location |
|---|---|
| Tet-responsive promoter P_{hCMV-1} | 7-439 |
| Tet responsive element (TRE) | |
| Location of seven tetO 19-mers | 7-319 |
| Fragment containing P_{min CMV} | 320-439 |
| TATAA box | 342-349 |
| HA tag | 505-537 |
| Multiple cloning site (MCS) | 546-600 |
| Fragment containing β-globin poly-A signal | 608-1774 |
| Fragment containing Col E1 origin of replication | 1975-2619 |
| Ampicillin resistance gene (β-lactamase) | |
| Start codon (ATG) | 3429-3427 |
| Stop codon | 2769-2767 |
| Hygromycin resistance gene | 3838-5392 |
| P_{SV40} promoter | 3838-4108 |
| Hygromycin coding sequence | 4175-5200 |
| SV40 poly-A signal | 5338-5392 |

The pTRE2hyg2-HA construct can be propagated in DH5a and other general purpose strains. The pTRE2hyg2-HA construct comprises a selectable marker by conferring ampicillin resistance (100 μg/ml) in E. coli hosts. The pTRE2hyg2-HA construct comprises an E. coli Co1 E1 origin of replication. A nucleic acid encoding RNF5 was cloned into the construct at the multiple cloning site.

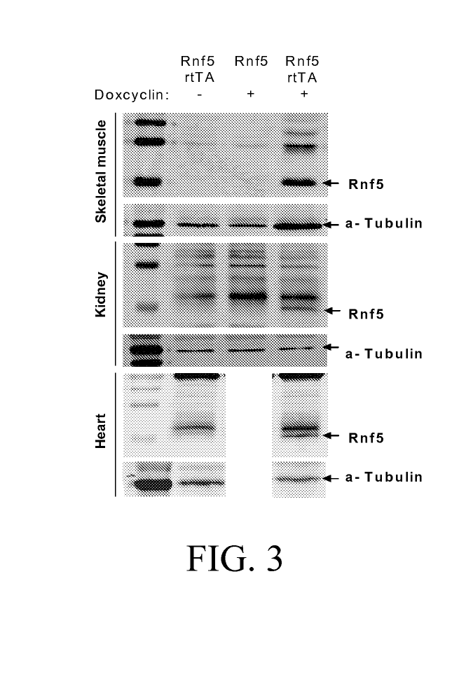

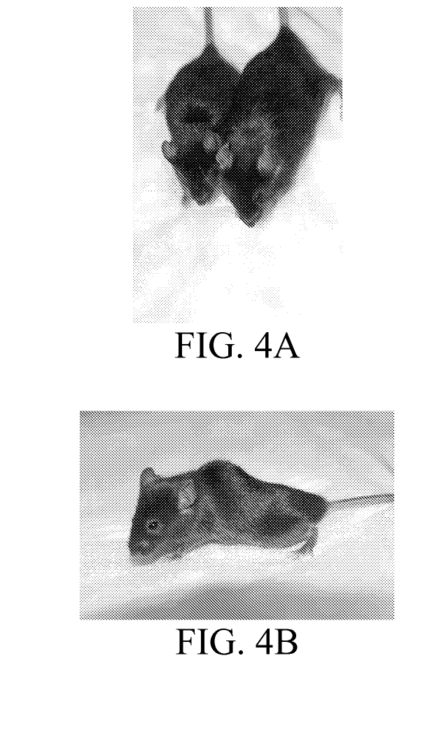

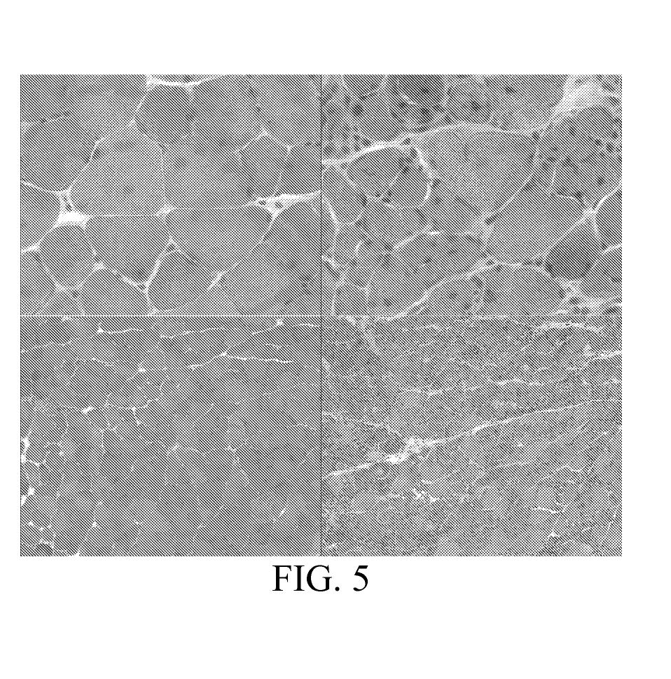

RNF5 Tg animals were allowed to develop in the absence of induced RNF5 expression. Dox was added to induce Tet expression in young adults (8-12 weeks after birth). Analysis of several organs confirmed elevated expression of RNF5 (FIG. 3), with the highest level of expression in skeletal muscle and to a lesser extent in heart. Striking phenotypes were observed in the mice in which RNF5 expression was induced. Primarily, we have noticed a decrease in body weight (30%) within 2 weeks after induction of RNF5 expression. Within this time frame, mice that were subjected to elevated RNF5 expression also exhibit a hunch back phenotype (FIG. 4), which was exuberated upon exercise (limited to walking on a rota-rode). The latter, also revealed that elevated expression of RNF5 results in rapid exhaustion and difficulties to run properly. Further exposure to Dox ultimately resulted in death within 7 weeks. The cause of death is associated with general weakness.

Analysis of different muscle types obtained from these mice revealed dramatic histological changes that resemble those seen in certain muscular dystrophies (FIG. 5). In all muscle types analyzed (Triceps, tibialis anterior, vas lat); a very important increase in regenerating fibers with central nuclei is evident and is associated with inflammatory infiltrates. However, no fibrotic tissues could be observed in these samples, suggesting that the regeneration process is still effective. The latter could be triggered by mechanical fiber destruction, due to RNF5 induced cytoskeletal disorganization, however, dystrophin glycoprotein complex is not altered, and other cytoskeletal structures can be affected. As observed in many muscular dystrophies, fiber hypertrophy associated with fiber splitting is present in the muscle section of mice expressing RNF5 transgene. Of importance, mice expressing the RNF5 in which the RING was mutated did not exhibit such phenotypes, indicating that the E3 ligase activity of RNF5 is required for changes seen in muscle organization and function. The absence of fibrosis and the rapid evolution of the disease seen upon RNF5 overexpression indicates a muscle wasting disorder or an atypical muscular dystrophy (rather than Duchenne of sarcoglycanopathy for which the mutant mice survive with mild phenotype for over a year).

Similar phenotypes were observed using an MCK promoter, which is muscle specific. These are Tet-off MCK mice which were crossed with the disclosed RNF5 mice and exhibit induced expression of RNF5 once Dox is removed from the drinking water.

### 2. Example 2

### The ER-Bound RING Finger Protein 5 (RNF5/RMA1) Causes Severe Muscle Disorder in Transgenic Mice and is Deregulated in Inclusion Body Myositis

i. Material and Methods

Generation of the RNF5 transgenic mice: The mouse isoform of the RNF5 gene was cloned by PCR in frame with the HA tag into the pTRE2-HA vector using MluI and NheI restriction sites and sequenced. The linear fragment resulting from a HpaI-SapI digestion was then used for pronuclear injection. After microinjection, the fertilized eggs were transferred into C57/B16 female recipients and crossed with C57/B16 males. Conditional RNF5 overexpression was achieved by crossing RNF5 Tg animals with rtTA Tg mice, expressing the tetracyclin responsive Transcriptional Activator under the control of the ubiquitous CMV-β-actin promoter and the genotypes were verified by PCR reaction using the following primers:

| | |
|---|---|
| RNF5-forward: | |
| GTACCCATACGATGTTCCAGATTACGC; | (SEQ ID NO: 6) |
| RNF5-reverse: | |
| CTGAGCAGCCAGAAAAAGAAAAAGATG; | (SEQ ID NO: 7) |
| rtTA-forward: | |
| CGGGTCTACCATCGAGGGCCTGCT; | (SEQ ID NO: 8) |
| and | |
| rtTA-reverse: | |
| CCCGGGGAATCCCCGTCCCCCAAC. | (SEQ ID NO: 9) |

Both RNF5 and rtTA transgenic lines were kept as heterozygous and maintained as separate lines by crossing with WT C57/BL6 animals.

Immunoblot and immunohistochemistry analysis: For expression analysis, frozen tissues were collected, flash frozen and pulverized using a mortar and pestle in liquid nitrogen. Proteins were extracted by resuspension in cold RIPA buffer containing anti-proteases and the extracts were homogenized and clarified by high speed centrifugation. The protein concentration in the supernatant was determined by Bradford assay. RNF5 expression was analyzed either by straight immunoblot or after immunoprecipation using an affinity purified RNF5 polyclonal antibody (1:2000 dilution). Expression for ER stress markers was assessed using rabbit Grp78 (Santa Cruz), rabbit Grp94 (abcam) and mouse PDI (Stressgen) antibodies and using GAPDH (Ambion) as a control.

For immunohistochemistry analysis, skeletal muscle tissues embedded in O.C.T. Compound (Sakura Tissue-Tek, product #4583) were pinned on cork pieces and snap-frozen in isopentane cooled in liquid nitrogen. Frozen tissues were stored at −80° C. until cross-sections (8 μm thick) were prepared using a Leica CM3050S cryostat and stained.

H&E stainings and Gomori-Trichrome are performed as previously described (Dubowitz V, Muscle Biopsy, London 1985). For immunostaining, sections were fixed in cold acetone for 5 minutes, permeabilized with 0.1% Triton X for 10 minutes, and blocked with 1% glycine for 30 minutes. Immunostainings were performed using dystrophin antibody (Vector clone Dy8/6C5, diluted 1:20), CD45 (BD Pharmingen, clone 30-F11, diluted 1:200), Cd11b (BD Pharmingen, clone M170, diluted 1:100), embMHC (hybridoma bank F1.652, diluted 1:3), myogenin (hybridoma bank F5D, diluted 1:100), laminin antibody (Sigma L9393, diluted 1:50), and RNF5 polyclonal antibody generated in the lab (dilution 1:100), paxillin (Upstate, diluted 1:50). For mouse monoclonal antibodies, the sections were incubated in biotinylated anti-mouse and avidin-conjugated fluorescein. (Mouse on Mouse Kit, Vector #FMK2201) For rat and rabbit antibodies, the sections were incubated with alexa fluor conjugated secondaries, diluted 1:600 in Dako antibody diluent (#S3022), for 1 hour at RT.

Images were captured using an Olympus IX71 fluorescence microscope and the Slidebook version 4.0 software.

Phenotypical Analysis of the double transgenic and control animals: A 2 mg/ml solution of doxycyclin supplemented with 5% sucrose was given to littermates animals between 12 to 24 weeks of age in the drinking water during 6 weeks. Phenotypic alteration (body mass, weakness, blood withdrawal) were observed on a bi-weekly basis and the animals were sacrificed after 6 weeks of treatment. Organs were individually weighted on a precision sale after trimming of the extra-tissues. Organs were then both flash frozen in liquid nitrogen for expression analysis and frozen in OTC for immunohistochemical analysis.

Morphometric analysis of skeletal muscle cross-sections: Cross-sections of the triceps brachii, tibialis anterior, and vastus lateralis muscles from the double transgenic animals and their matching controls were immunostained with dystrophin and H&E to delineate the fiber and the cross section area of each fiber was quantified using the Scion software version 4.0.3.2. (NIH).

Evans Blue Dye Injection: Evans Blue Dye (SIGMA) was diluted in PBS (10 mg/ml) and filter-sterilized and the dye was injected through the tail vein at a concentration of 100 μl per gram of body weight. 16 hours after injection, the mice were sacrificed and the dissected muscles inspected for the presence of the blue dye in the muscle. Skeletal muscles were then fresh-frozen and cross-sections were further analyzed for the presence of the dye and counter-stained with dystrophin antibody as described previously.

Serum Creatine Kinase Assay: 150 μl of blood was collected in heparin treated tubes from periorbital bleeding and the serum fraction was extracted by double centrifugation for 5 minutes at 5000 rpm. Creatine kinase levels were monitored using CPK-NAC kit (JAS Diagnostics #CPK1-15) and analyzed on a Roche Cobas Mira classic apparatus.

ii. Results

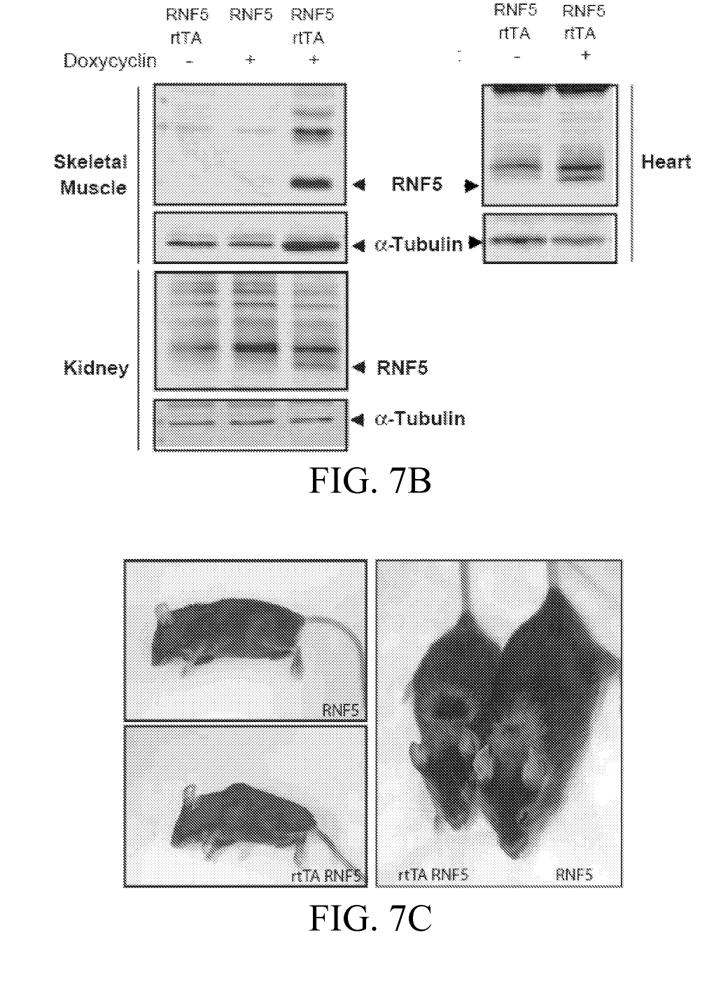

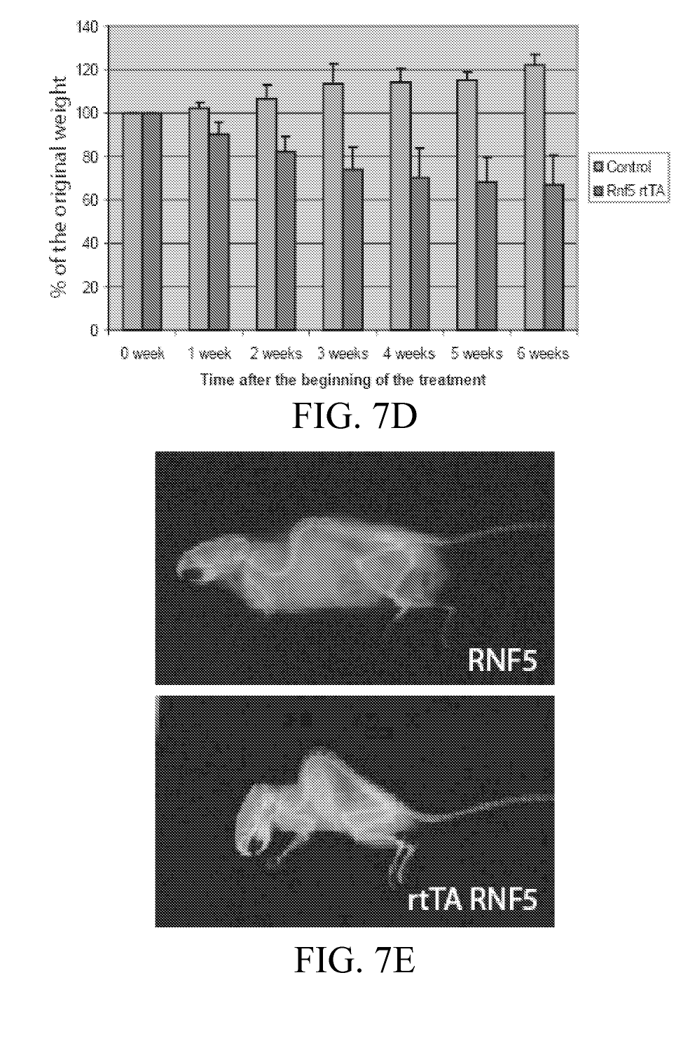

Construction of RNF5 transgenic mouse: To generate an inducible RNF5 expression system in mice, a construct containing the RNF5 gene was cloned downstream of the minimal Tet-ON operator (Tetracyclin Responsive Element (TRE) containing promotor). RNF5 transgenic (Tg) lines were then established by pronuclear injection and implantation in C57/B16 recipient mice. The RNF5 rtTA double transgenic animals (DTg) and their corresponding control littermates (RNF5 STg) were generated by crossing RNF5 Tg with rtTA animals, expressing the tetracyclin responsive Transcriptional Activator under the control of the ubiquitous CMV-β-actin promotor (FIG. 7A) (Wiekowski, M. T., et al., 2004).

Over-expression of RNF5 protein was confirmed in different organs of the RNF5 rtTA DTg animals provided with doxycyclin in the drinking water (FIG. 7B). Expression of RNF5 protein was evident in doxycyclin treated double transgenic animals as early as 4 days after addition, but not in doxycyclin treated single transgenic animals (RNF5 STg) or untreated double transgenic animals. The RNF5 transgene is expressed at different levels depending on the organs analyzed, with the higher level of RNF5 expression seen in skeletal muscle, heart and to a lesser extent in kidney (FIG. 7B). Conversely, RNF5 levels were very low in the liver and undetectable in several tissues including the brain, the lungs, the spleen and the kidneys, which is consistent with the low transcriptional levels of the rtTA activator and the differential expression pattern described for the rtTA transgene (Wiekowski, M. T., et al., 2004). These data indicate that RNF5 expression is tightly controlled in the DTg mice and that the system is not subjected to transcriptional leakage in the absence of doxycyclin induction. Furthermore, the prominent expression of RNF5 in skeletal muscles makes it a suitable system to study its function in this organ.

Induction of RNF5 transgene leads to rapid weight loss and early onset of muscle wasting and kyphosis: Double transgenic but not control animals subjected to doxycyclin treatment exhibited a significant weight loss as early as the 2 week time point (FIG. 7D). By 4 weeks, clear phenotypic differences were evident between the double transgenic animals and their control littermates including a significant decrease in body mass and visible kyphosis (FIG. 7C, 7D). After 5-6 weeks of overexpressing of RNF5, the phenotype was even more severe: the animals showed decreased activity and exhibited pelvic limb weakness. Death occurred 7 weeks following initiation of the doxycyclin treatment. Radiographic analysis did not identify a skeletal abnormality in the experimental or control groups, indicating that kyphosis was associated with muscle weakness (FIG. 7E). To confirm this, skeletal muscles from the double transgenic mice were extracted and analyzed for histopathology.

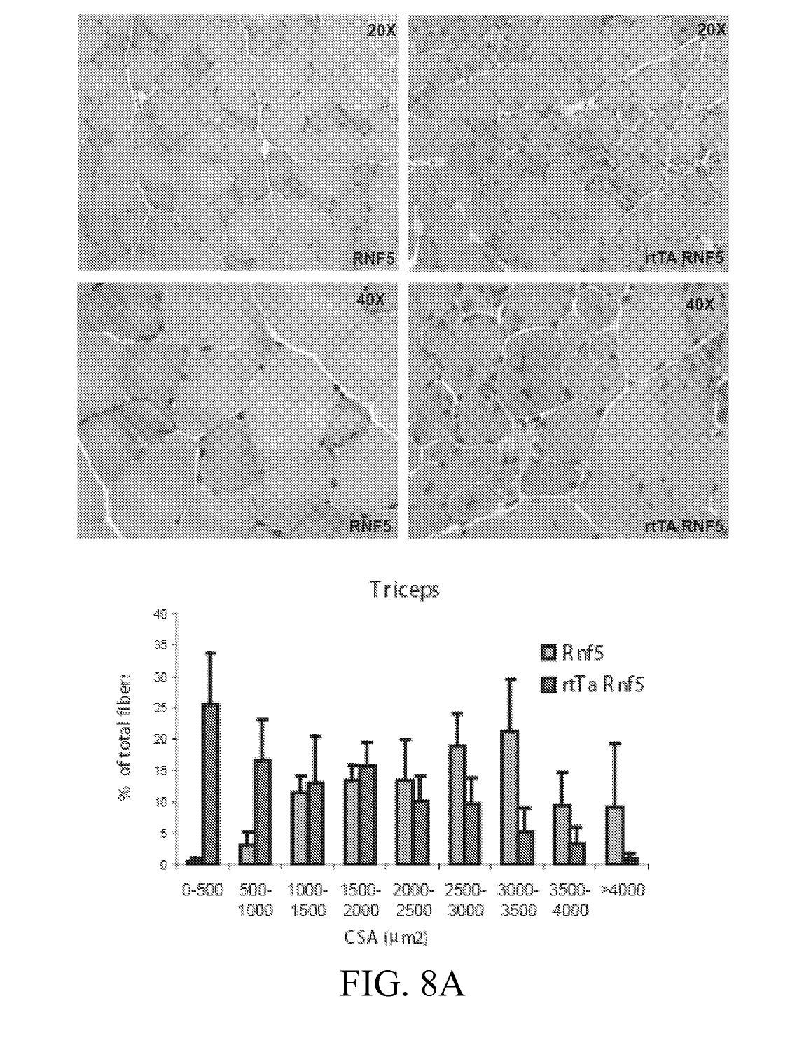

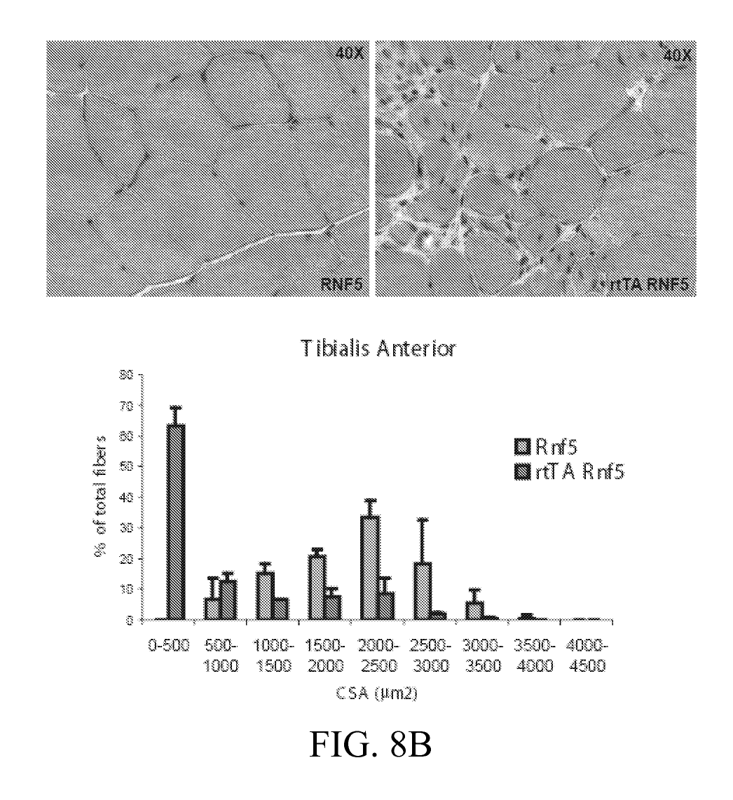

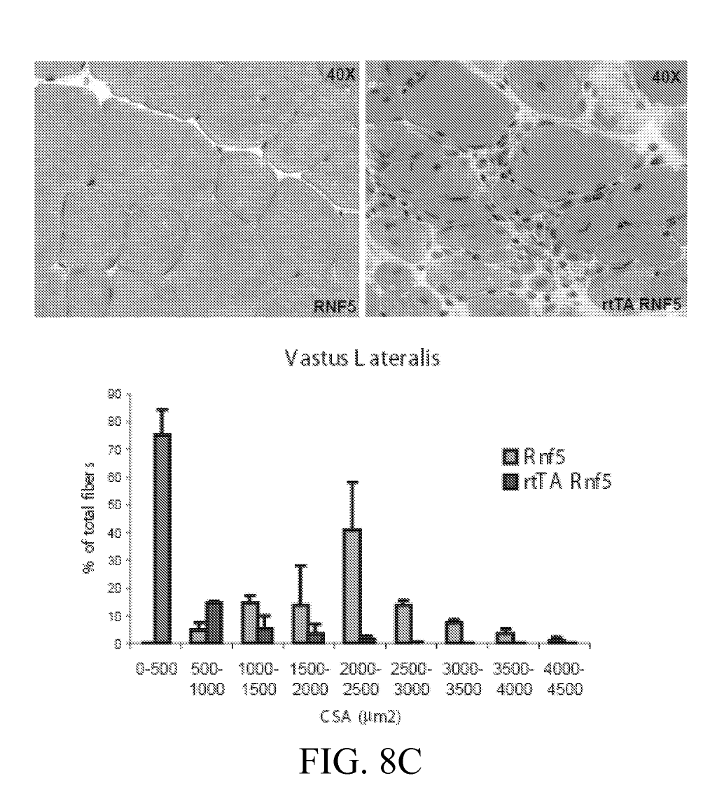

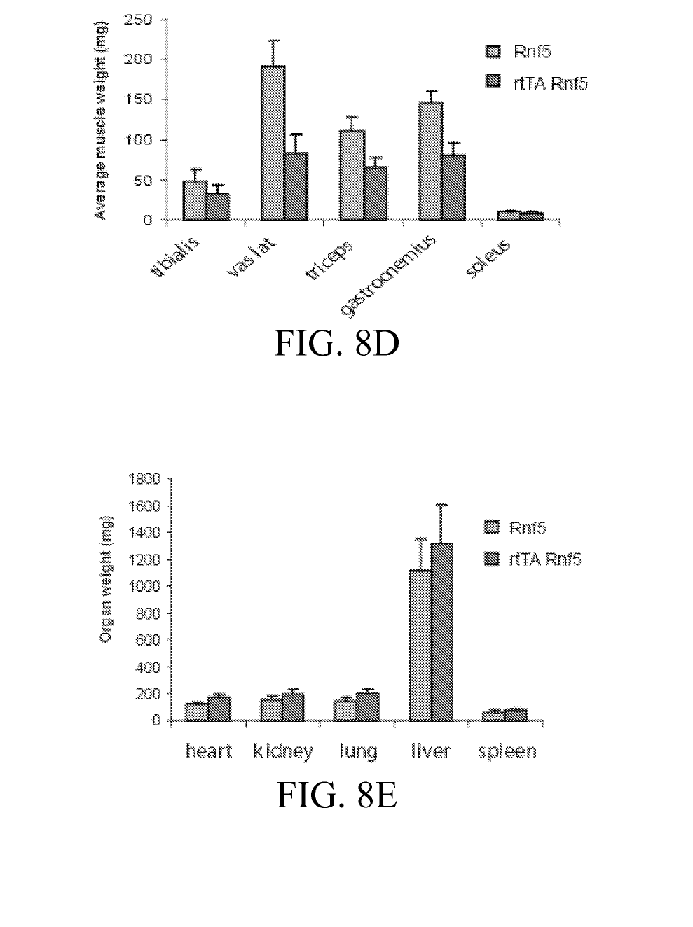

Compared to controls, double transgenic animals exhibited clear pathological changes in all skeletal muscles analyzed (FIG. 8A-C). In transverse sections of fresh frozen specimens from the triceps brachii, tibialis anterior, and vastus lateralis muscles, there was a marked variability in myofiber size in the treated double transgenic animals with numerous small caliber fibers having a round shape and multifocal clusters of mononuclear cells. As there was no elevation of atrogin-1 in the muscle from the double transgenic animals, the decrease in fiber size was not likely a result of muscle atrophy. Many muscle fibers of both small and larger caliber contained internal nuclei. Measurement of the cross-sectional area confirmed the presence of many small fibers, most markedly within the vastus lateralis muscle (FIG. 8C). The small size of the muscle fibers was reflected by a decrease in the weight of each muscle (FIG. 8D). Loss of muscle mass may account for the global decrease in body weight observed in the double transgenic animals, as the weight of the other organs was not affected (FIG. 8E). Among the different muscles analyzed, the vastus lateralis was the most affected, demonstrated both by the decrease in cross sectional area measurement and the more pronounced weight loss.

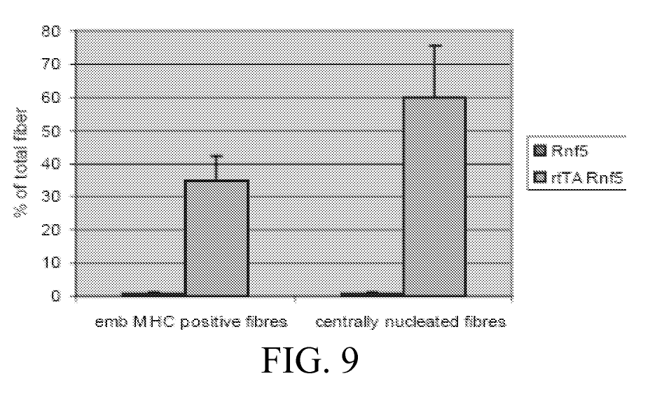

RNF5 overexpression is associated with increased muscle degeneration-coupled regeneration: Quantitative analysis revealed that 50% of the muscle fibers of RNF5 rtTA DTg, but not control animals, are centrally nucleated, and that 30% of the small fibers stain positively for embryonic myosin heavy chain (emb MHC), a known marker for early muscle regeneration (FIG. 9). Consistent with this observation, positive staining for myogenin transcription factor was also observed in the small myofibers of RNF5 rtTA DTg animal muscle, but not in their control littermates. Myogenin is expressed during differentiation of activated satellite cells in muscle fibers and therefore further underscores the ongoing regenerative process (Charge, S. B. and M. A. Rudnicki, 2004). These data indicate that expression of RNF5 induces extensive muscle fiber regeneration.

Clusters of mononuclear cells, staining positive with antibodies against the pan-leukocyte marker CD45 and macrophage marker CD11b, were observed at sites of muscle regeneration. The pattern and type of cellular infiltration is consistent with previous degeneration and clearing of necrotic debris. Notably, lymphocytic infiltration was not observed and fibrosis was not a feature. These data indicate that overexpression of RNF5 results in myofiber degeneration-coupled regeneration.

Increased creatine kinase (CK) levels is associated with myodegeration in RNF5 rtTA DTg mice: Myodegeneration is associated with elevations in the serum CK concentration. This is particularly noted in the muscular dystrophies where mutations in the dystrophin-glycoprotein complex (DCG) result in defects in sarcolemmal integrity and markedly elevated serum CK concentrations. To assess whether the dystrophy like phenotype found in the RNF5 rtTA DTg animals is due to impaired sarcolemmal integrity, changes in serum CK levels and staining of muscle tissue were monitored following injection with Evans Blue dye (EBD). Elevated levels of serum CK were found in the RNF5 rtTA DTg animals, but not in their control littermates, after 6 weeks of doxycyclin treatment. This finding points to the presence of myofiber damage. To visualize the extent of fiber damage, EBD was injected into the RNF5 rtTA DTg and their control littermates.

Contrary to their matching controls, the skeletal muscle of the RNF5 rtTA DTg animals exhibited positive staining for EBD within a number of fibers, consistent with degeneration (myonecrosis) and elevated serum CK levels in these mice. In agreement with these observations, EBD positive muscle fibers were negative for dystrophin staining and surrounded by immune cells positive for CD45 and CD11b staining. Immunostaining using markers for DGC proteins including dystrophin and alpha-sarcoglycan did not show any major difference between DTg and control cross-sections. Consistently, no alteration in the sarcomeric units could be observed after ultrastructural analysis. Further, in vitro differentiation of primary myoblasts cultured from the double transgenic animals treated with doxycyclin prior and during differentiation, did not reveal changes in the sarcomeric structure as depicted by staining for alpha-sarcomeric actinin.

This finding demonstrates that myofiber degeneration and necrosis account for the elevated levels of serum CK associated with RNF5 overexpression but that myodegeneration is not a primary inflammatory process nor is it a consequence of the alteration of common sarcolemmal structural proteins.

Therefore, unlike the currently available mouse models for muscular dystrophy, over-expression of RNF5 leads to a dystrophic like phenotype independently of the DGC complex. Thus, provided herein is a mouse model of muscular dystrophy that is independent of the dystrophin-glycoprotein complex (DGC).

Myofiber degeneration-coupled regeneration in RNF5 rtTA DTg mice is associated with altered ERAD chaperones: Given that RNF5 is a membrane-anchored protein which is primarily localized to the endoplasmic reticulum (ER) and implicated in recognition of misfolded proteins, the possibility that RNF5 over-expression could affect the ERAD or ER stress response was examined. Consistent with this, RNF5 exhibited a dense perinuclear staining in the endoplasmic reticulum of mature and regenerative fibers. Notably, RNF5 staining was higher in the regenerative fibers and along the nascent sarcoplasmic reticulum network inside the fiber.

ER stress is commonly considered to be a protective response. However, high levels of ER stress or ER dysfunction may also impair cell survival by triggering apoptotic pathways. Therefore, the onset of ER stress could cause fiber death with concomitant induction of muscle regeneration, without directly affecting the muscle structural components (Kaufman, R. J., 2002; Nakanishi, K., et al., 2005; Tarricone, E., et al., 2006). However, analysis of TUNEL and cleaved caspase 3 as markers for apoptosis did not reveal any changes in programmed cell death. Similarly, no changes were observed in the levels of CHOP and cleaved caspase 12. These data indicate that the degeneration process observed in RNF5 rtTA DTg animals is not linked with ER-associated programmed cell death.

Nevertheless, various stages of myonecrosis were evident in areas of regeneration as IgG stained fibers, consistent with the presence of EBD stained fibers, and clusters of macrophages clearing necrotic myofiber debris. These changes are likely to represent the consequence of degeneration-coupled regeneration phenotype induced by RNF5.

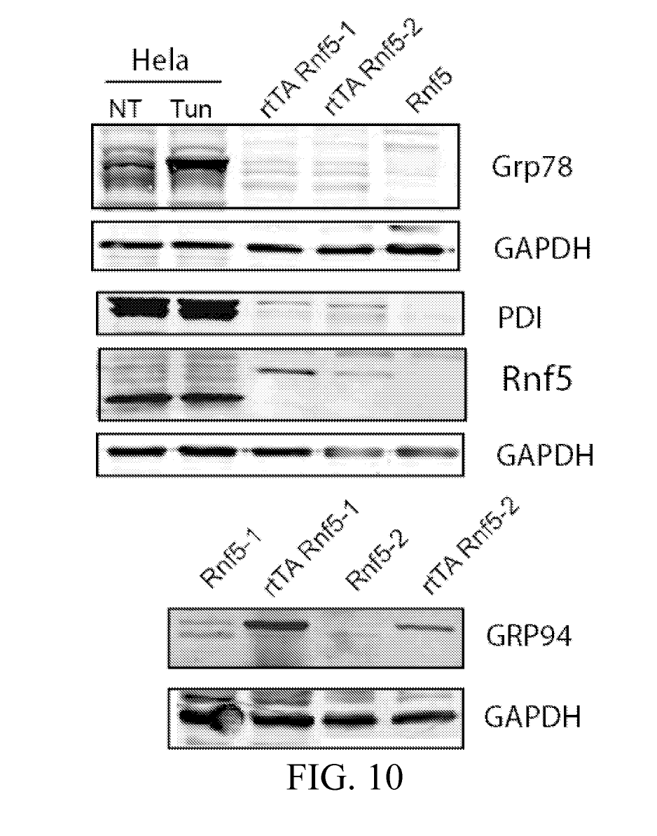

Given the association of RNF5 with the ER membrane (Younger, J. M., et al., 2006) and its role in quality control of ERAD the levels of different types of ER stress markers were next analyzed in the muscle extract form the RNF5 rtTA DTg animals. A clear and consistent, albeit moderate, increase in the expression of PDI, Grp78 and Grp94, was seen in RNF5 rtTA DTg but not the control mice (FIG. 10). Grp94 upregulation was confirmed by immunohistochemistry on the DTg muscle sections. As these chaperones are important in the regulation of ER stress, these findings indicate that RNF5 overexpression impact the ER stress response.

Intriguingly, in addition to elevated expression, a change in Grp94 migration pattern was also noted in RNF5 rtTA DTg, but not in their matching control. Grp94 has been previously reported to be important for myoblast fusion (Gorza, L. and M. Vitadello, 2000), a critical step in regenerating muscle. Change in Grp94 localization is expected to impact its contribution to myoblast fusion and therefore impact regeneration process, consistent with the observations seen in the RNF5 DTg muscles (Tarricone, E., et al., 2006). These findings indicate that RNF5 exerts its effect on muscle physiology by modulating the expression and localization, thereby affecting the function of specific components of the ER, including Grp94.

Aberrant RNF5 expression in human myopathies: Based on the finding with the RNF5 rtTA DTg mice, it was next assessed whether human myopathies can exhibit deregulated expression or localization of RNF5. Hela cells were transfected with a plasmid containing a Chemy marker and expressing RNF5 specific shRNA or its scramble version. Cells were stained using RNF5 antibody after paraformaldehyde (PFA) fixation and Triton permeabilization. The following sequence was cloned into the pSUPER-RETRO vector which was used to generate stable clones in which RNF5 expression was inhibited:

(SEQ ID NO: 10)

5′-GATCCCCAGCTGGGATCAGCAGAGAGTTCAAGAGACTCTCTGCTGAT
CCCAGCTTTTTTGGAAA-3′.

In addition, the following sequences were used for transient transfection experiments to inhibit RNF5 expression:

| | |
|---|---|
| 5′-UGUCUUCAUCAGUGGCUGGtt-3′ | (SEQ ID NO: 11) |
| 5′-GUAUGUAAAGCUGGGAUCAtt-3′, | (SEQ ID NO: 12) |
| and | |
| 5′-GCUGGGAUCAGCAGAGAGAtt-3′. | (SEQ ID NO: 13) |

Representative human myopathies were screened for possible changes in the pattern or level of RNF5 expression using RNF5 antibodies with confirmed specificity. Common muscular dystrophies that are associated with dysfunction of DGC, such as Duchenne or Becker forms of muscular dystrophy, did not exhibit any alteration in the pattern of RNF5 expression. However, analysis of biopsies from sporadic forms of IBM (sIBM) revealed that RNF5 is mislocalized to intrafiber aggregates, compared with the normal muscle controls. Also generated were RNF5 mutant Tg mice, wherein the RING finger domain was mutated so that ubiquitin ligase activity of the protein was no longer available. These mice were treated similar to the WT, but did not generate muscle phenotypes as seen with the WT animals.

To determine if changes in RNF5 localization was a common feature of the IBM type of degenerative diseases, RNF5 staining was also evaluated in muscle cross-sections of a mouse model for hereditary IBM, DMRV (Distal Myopathy with Rimmed vacuoles) (Malicdan, M. C., et al., 2007). A strong increase of RNF5 staining in fibers containing rimed vacuoles was also observed in transgenic animals compared to their control littermates. These finding reveal that altered RNF5 expression is seen in specific subtypes of muscular diseases which are associated with dysfunction of the ER or the ERAD machinery.

### G. References

Askanas, V. and W. K. Engel, Inclusion-body myositis: a myodegenerative conformational disorder associated with Abeta, protein misfolding, and proteasome inhibition. Neurology, 2006. 66(2 Suppl 1): p. S39-48.
Bodine, S. C., et al., Identification of ubiquitin ligases required for skeletal muscle atrophy. Science, 2001. 294(5547): p. 1704-8.
Boonyarom, O. and K. Inui, Atrophy and hypertrophy of skeletal muscles: structural and functional aspects. Acta Physiol (Oxf), 2006. 188(2): p. 77-89.
Broday, L., I. Kolotuev, C. Didier, A. Bhoumik, B. Podbilewicz, and Z. Ronai, The LIM domain protein UNC-95 is required for the assembly of muscle attachment structures and is regulated by the RING finger protein RNF-5 in C. elegans. J Cell Biol, 2004. 165(6): 857-67.
Cao, P. R., H. J. Kim, and S. H. Lecker, Ubiquitin-protein ligases in muscle wasting. Int J Biochem Cell Biol, 2005. 37(10): 2088-97.
Charge, S. B. and M. A. Rudnicki, Cellular and molecular regulation of muscle regeneration. Physiol Rev, 2004. 84(1): p. 209-38.
Didier, C., L. Broday, A. Bhoumik, S. Israeli, S. Takahashi, K. Nakayama, S. M. Thomas, C. E. Turner, S. Henderson, H. Sabe, and Z. Ronai, RNF5, a RING finger protein that regulates cell motility by targeting paxillin ubiquitination and altered localization. Mol Cell Biol, 2003. 23(15): 5331-45.
Durbeej M., Campbell K P. Muscular dystrophies involving the dystrophin-glycoprotein complex: an overview of current mouse models Current Opin. Genet. Dev. (2002) 3:349-61
Fielitz, J., et al., Loss of muscle-specific RING-finger 3 predisposes the heart to cardiac rupture after myocardial infarction. Proc Natl Acad Sci USA, 2007. 104(11): p. 4377-82.
Frosk, P., et al., Limb-girdle muscular dystrophy type 2H associated with mutation in TRIM32, a putative E3-ubiquitin-ligase gene. Am J Hum Genet, 2002. 70(3): p. 663-72.
Glass, D. J., Signalling pathways that mediate skeletal muscle hypertrophy and atrophy. Nat Cell Biol, 2003. 5(2): p. 87-90.
Gorza, L. and M. Vitadello, Reduced amount of the glucose-regulated protein GRP94 in skeletal myoblasts results in loss of fusion competence. Faseb J, 2000. 14(3): p. 461-75.
Hishiya, A., et al., A novel ubiquitin-binding protein ZNF216 functioning in muscle atrophy. Embo J, 2006. 25(3): p. 554-64.
Hoffman, E. P., R. H. Brown, Jr., and L. M. Kunkel, Dystrophin: the protein product of the Duchenne muscular dystrophy locus. Cell, 1987. 51(6): p. 919-28.
Jackman, R. W. and S. C. Kandarian, The molecular basis of skeletal muscle atrophy. Am J Physiol Cell Physiol, 2004. 287(4): p. C834-43.
Kaufman, R. J., Orchestrating the unfolded protein response in health and disease. J Clin Invest, 2002. 110(10): p. 1389-98.
Kobinger G P, Louboutin J P, Barton E R, Sweeney H L, Wilson J M. Correction of the dystrophic phenotype by in vivo targeting of muscle progenitor cells. Hum. Gene Ther. (2003) 14: 1441-9.
Kudryashova, E., D. Kudryashov, I. Kramerova, and M. J. Spencer, Trim32 is a Ubiquitin Ligase Mutated in Limb Girdle Muscular Dystrophy Type 2H that Binds to Skeletal Muscle Myosin and Ubiquitinates Actin. J Mol Biol, 2005. 354(2): 413-24.
Kyushiki, H., Y. Kuga, M. Suzuki, E. Takahashi, and M. Horie, Cloning, expression and mapping of a novel RING-finger gene (RNF5), a human homologue of a putative zinc-finger gene from Caenorhabditis elegans. Cytogenet Cell Genet, 1997. 79(1-2): 114-7.
Li S, Kimura E, Fall B M, Reyes M, Angello J C, Welikson R, Hauschka S D, Chamberlain J S. Stable transduction of myogenic cells with lentiviral vectors expressing a minidystrophin. Gene Therapy (2005) 12, 1099-1108.
Malicdan, M. C., et al., A Gne knockout mouse expressing human V572L mutation develops features similar to distal myopathy with rimmed vacuoles or hereditary inclusion body myopathy. Hum Mol Genet, 2007. 16(2): p. 115-28.
Manfra, D. J., S. C. Chen, K. K. Jensen, J. S. Fine, M. T. Wiekowski, and S. A. Lira, Conditional expression of murine Flt3 ligand leads to expansion of multiple dendritic cell subsets in peripheral blood and tissues of transgenic mice. J Immunol, 2003. 170(6): 2843-52.
Matsuda, N. and A. Nakano, RMA1, an Arabidopsis thaliana gene whose cDNA suppresses the yeast sec15 mutation, encodes a novel protein with a RING finger motif and a membrane anchor. Plant Cell Physiol, 1998. 39(5): 545-54.
Matsuda, N., T. Suzuki, K. Tanaka, and A. Nakano, Rma1, a novel type of RING finger protein conserved from Arabidopsis to human, is a membrane-bound ubiquitin ligase. J Cell Sci, 2001. 114: 1949-57.
McElhinny, A. S., et al., Muscle-specific RING finger-2 (MURF-2) is important for microtubule, intermediate filament and sarcomeric M-line maintenance in striated muscle development. J Cell Sci, 2004. 117(Pt 15): p. 3175-88.
Nagaraju, K., et al., Activation of the endoplasmic reticulum stress response in autoimmune myositis: potential role in muscle fiber damage and dysfunction. Arthritis Rheum, 2005. 52(6): p. 1824-35.
Nagaraju, K., et al., Conditional up-regulation of MHC class I in skeletal muscle leads to self-sustaining autoimmune myositis and myositis-specific autoantibodies. Proc Natl Acad Sci USA, 2000. 97(16): p. 9209-14.
Nakanishi, K., T. Sudo, and N. Morishima, Endoplasmic reticulum stress signaling transmitted by ATF6 mediates apoptosis during muscle development. J Cell Biol, 2005. 169(4): p. 555-60.
Nakayama, K., Frew, I. J., Hagensen, M., Skals, M., Habelhah, H., Erdjument-Bromage, H., Tempst, P., Frappell, P. B., Bowtell, D. D., and Ronai, Z. Siah2 regulates stability of prolyl-hydroxylases, controls HIF1α abundance and modulates physiological responses to hypoxia. Cell (2004) 117, 941-952.
Nastasi, T., A. Bongiovanni, Y. Campos, L. Mann, J. N. Toy, J. Bostrom, R. Rottieri, C. Hahn, J. W. Conaway, A. J. Harris, and A. D'Azzo, Ozz-E3, a muscle-specific ubiquitin ligase, regulates beta-catenin degradation during myogenesis. Dev Cell, 2004. 6(2): 269-82.
Reid, M. B., Response of the ubiquitin-proteasome pathway to changes in muscle activity. Am J Physiol Regul Integr Comp Physiol, 2005. 288(6): p. R1423-31.
Schoser, B. G., et al., Commonality of TRIM32 mutation in causing sarcotubular myopathy and LGMD2H. Ann Neurol, 2005. 57(4): p. 591-5.
Tarricone, E., et al., Cell stress response in skeletal muscle myofibers. Ann N Y Acad Sci, 2006. 1069: p. 472-6.
Tisdale, M. J., Biology of cachexia. J Natl Cancer Inst, 1997. 89(23): p. 1763-73.
Vattemi, G., et al., Endoplasmic reticulum stress and unfolded protein response in inclusion body myositis muscle. Am J Pathol, 2004. 164(1): p. 1-7.
Watanabe, N. and C. Higashida, Formins: processive cappers of growing actin filaments. Exp Cell Res, 2004. 301(1): 16-22.
Watts, G. D., et al., Inclusion body myopathy associated with Paget disease of bone and frontotemporal dementia is caused by mutant valosin-containing protein. Nat Genet, 2004. 36(4): p. 377-81.
Wiekowski, M. T., et al., Disruption of neutrophil migration in a conditional transgenic model: evidence for CXCR2 desensitization in vivo. J Immunol, 2001. 167(12): p. 7102-10.
Witt, S. H., et al., MURF-1 and MURF-2 target a specific subset of myofibrillar proteins redundantly: towards understanding MURF-dependent muscle ubiquitination. J Mol Biol, 2005. 350(4): p. 713-22.
Younger, J. M., et al., Sequential quality-control checkpoints triage misfolded cystic fibrosis transmembrane conductance regulator. Cell, 2006. 126(3): p. 571-82.
Zengel, J. M. and H. F. Epstein, Mutants altering coordinate synthesis of specific myosins during nematode muscle development. Proc Natl Acad Sci USA, 1980. 77(2): 852-6.

What is claimed is:

1. A non-human transgenic animal wherein nucleated cells of the animal comprise a nucleic acid encoding RNF5 operably linked to an expression control sequence, wherein expression of the RNF5 encoded by the nucleic acid differs from native expression of RNF5 in the cells.

2. The animal of claim 1, wherein the expression control sequence is not operably linked to the nucleic acid encoding RNF5 in nature.

3. The animal of claim 1, wherein the nucleic acid encoding RNF5 is heterologous to the animal.

4. The animal of claim 1, wherein transgenic expression of RNF5 results in muscle wasting in the animal.

5. The animal of claim 1, wherein the RNF5 is expressed in muscle cells.

6. The animal of claim 1, wherein the expression control sequence comprises a cell-specific promoter.

7. The animal of claim 6, wherein the cell-specific promoter is a muscle creatine kinase (NICK) promoter, desmin promoter, or myoglobin promoter.

8. The animal of claim 1, wherein the expression control sequence comprises an inducible promoter.

9. The animal of claim 8, wherein the nucleated cells further comprise a transgene encoding a transactivator protein, wherein the transactivator protein conditionally induces expression of the transgene encoding RNF5.

10. The animal of claim 9, wherein inducible expression by the transactivator protein is conditioned on the presence of tetracycline or derivative thereof.

11. The animal of claim 1, wherein the animal is selected from the group consisting of avian, bovine, canine, caprine, equine, feline, leporine, murine, ovine, porcine, primate.

12. The animal of claim 1, wherein the animal is a mouse, dog or cat.

13. The animal of claim 1, wherein the nucleic acid comprises the nucleic acid sequence set forth in SEQ ID NO:1.

14. The animal of claim 1, wherein the nucleic acid has at least 95% sequence identity to the nucleic acid sequence set forth in SEQ ID NO:1, wherein the polypeptide has ubiquitin ligase activity.

15. The animal of claim 1, wherein the nucleic acid hybridizes under stringent conditions to a hybridization probe consisting of the nucleic acid sequence set forth in SEQ ID NO:1.

16. The animal of claim 1, wherein the nucleic acid encodes a polypeptide comprising an amino acid having the sequence set forth in SEQ ID NO:2.

17. The animal of claim 1, wherein the nucleic acid encodes a polypeptide having at least 95% sequence identity to an amino acid having the sequence set forth in SEQ ID NO:2, wherein the polypeptide has ubiquitin ligase activity.

18. A method for producing the non-human transgenic animal of claim 1 comprising:
(a) introducing a nucleotide sequence encoding RNF5 operably linked to an expression control sequence into a fertilized animal oocyte;
(b) allowing the fertilized animal oocyte to develop to term; and
(c) identifying a transgenic animal whose genome comprises the nucleotide sequence encoding RNF5, wherein expression of the RNF5 results in muscle wasting in the animal.

19. A transgenic non-human animal having a phenotype characterized by altered expression of RNF5 polypeptide, the phenotype being conferred by a transgene contained in the cells of the animal, the transgene comprising a nucleic acid sequence which encodes RNF5 polypeptide.

20. The animal of claim 19, wherein the cells are somatic cells.

21. The animal of claim 19, wherein the cells are germ cells.

22. A method of treating or preventing muscle wasting in a subject, wherein the subject has elevated levels of RNF5 in the muscle, comprising administering to the subject a modulator of RNF5.

23. The method of claim 22, wherein the modulator of RNF5 is a functional nucleic acid.

24. The method of claim 22, wherein the subject has been diagnosed with muscular dystrophy.

25. A method of screening for an agent for use in treating or preventing muscle wasting, comprising:
(a) administering a candidate agent to a sample comprising RNF5,
(b) monitoring the sample for changes in the expression of RNF5, RNF5 ligase activity, degradation of an RNF5 substrate, or binding of RNF5 to a substrate,
wherein a detectable change in any one or more of these activities indicates that the candidate can be used for treating or preventing muscle wasting.

26. The method of claim 25, wherein the RNF5 is expressed in the sample by a cell comprising a nucleic acid encoding RNF5 operably linked to an expression control sequence.

27. The method of claim 25, wherein the sample comprising RNF5 is a non-human transgenic animal wherein nucleated cells of the animal comprise a nucleic acid encoding RNF5 operably linked to an expression control sequence, wherein expression of the RNF5 encoded by the nucleic acid differs from native expression of RNF5 in the cells.

28. A method for diagnosing a muscle wasting disease in a subject, comprising detecting RNF5 levels or activity in a sample from the muscle of the subject, wherein high levels or activity of RNF5 as compared to a control indicates muscle wasting in the subject.

29. A method of identifying a subject at risk for muscle wasting disease, comprising detecting RNF5 levels or activity in a sample from the muscle of the subject, wherein high levels or activity of RNF5 as compared to a control indicates a risk for muscle wasting in the subject.

30. A method of assessing the severity of muscle wasting in a subject, comprising detecting RNF5 levels or activity in a sample from the muscle of the subject, wherein the levels or activity of RNF5 as compared to a control correlate with the severity of muscle wasting in the subject.

31. A method of identifying targets of RNF5, comprising
(a) detecting binding of a candidate substrate to RNF5 in a sample comprising RNF5 as compared to a control lacking RNF5, or
(b) detecting degradation of the candidate substrate in a sample comprising RNF5 as compared to a control lacking RNF5,
wherein detectable binding of the candidate substrate to RNF5 or detectable degradation of the candidate substrate in the presence of RNF5 indicates that the candidate substrate is a target of RNF5.

32. A method of making an agent that modulates RNF5 activity, comprising
(a) administering a candidate agent to a sample comprising RNF5,
(b) monitoring the sample for changes in the expression of RNF5, RNF5 ligase activity, degradation of an RNF5 substrate, or binding of RNF5 to a substrate,
wherein a detectable change in any one or more of these activities indicates that the candidate agent modulates RNF5 activity,
(c) making the candidate agent indicated to modulate RNF5 activity.


SEQUENCE LISTING (13 sequences)

SEQ ID NO: 1 - DNA, 543 bp, Artificial Sequence
  Description of Artificial Sequence  note = synthetic construct
atggcagcagcggaggaggaggacgggggccccgaagggccaaatcgcgagcggggcggggcgggcgcgaccttcgaatgtaatatatgtttggagactgctcgggaagctgtggtcagtgtgtgtggccacctgtactgttggccatgtcttcatcagtggctggagacacggccagaacggcaagagtgtccagtatgtaaagctgggatcagcagagagaaggttgtcccgctttatgggcgagggagccagaagccccaggatcccagattaaaaactccaccccgcccccagggccagagaccagctccggagagcagagggggattccagccatttggtgataccgggggcttccacttctcatttggtgttggtgcttttccctttggctttttcaccaccgtcttcaatgcccatgagcctttccgccggggtacaggtgtggatctgggacagggtcacccagcctccagctggcaggattccctcttcctgtttctcgccatcttcttctttttttggctgctcagtatttga

SEQ ID NO: 2 - protein, 180 aa, Artificial Sequence
  Description of Artificial Sequence  note = synthetic construct
MAAAEEEDGGPEGPNRERGGAGATFECNICLETAREAVVSVCGHLYCWPCLHQWLETRPERQECPVCKAGISREKVVPLYGRGSQKPQDPRLKTPPRPQGQRPAPESRGGFQPFGDTGGFHFSFGVGAFPFGFFTTVFNAHEPFRRGTGVDLGQGHPASSWQDSLFLFLAIFFFFWLLSI

SEQ ID NO: 3 - DNA, 1053 bp, Artificial Sequence
  Description of Artificial Sequence  note = synthetic construct
ggccatggcagcagcggaggaggaggacgggggccccgaagggccaaatcgcgagcggggcggggcgggcgcgaccttcgaatgtaatatatgtttggagactgctcgggaagctgtggtcagtgtgtgtggccacctgtactgttggccatgtcttcatcagtggctggagacacggccagaacggcaagagtgtccagtatgtaaagctgggatcagcagagagaaggttgtcccgctttatgggcgagggagccagaagccccaggatcccagattaaaaactccaccccgcccccagggccagagaccagctccggagagcagagggggattccagccatttggtgataccgggggcttccacttctcatttggtgttggtgcttttccctttggctttttcaccaccgtcttcaatgcccatgagcctttccgccggggtacaggtgtggatctgggacagggtcacccagcctccagctggcaggattccctcttcctgtttctcgccatcttcttctttttttggctgctcagtatttgagctatgtctgcttcctgcccacctccagccagagaagaatcagtattgagggtccctgctgacccttccgtactcctggacccccttgacccctctatttctgttggctaaggccagccctggacattgtccaggaaggcctggggaggaggagtgaagtctgtgcatagatgggagagccttctgctcagaggctcactcagtaacgttgtttaattctctgccctggggaaggaggatggattgagagaatgtctttctcctctcctaagtctttgctttccctgatttcttgatttgatcttcaaaggtgggcaaagttccctctgactcttcccccactccccatcttactgatttaatttaatttttcactccccagagtctaatatggattctgactcttaagtgcttccgccccctcactacctcctttaatacaaattcaataaaaaaggtgaaatataaaaaaaaaaaaaaaaaaaaaaaaaaaaaaaaaaaaaaaa

SEQ ID NO: 4 - DNA, 2594 bp, Artificial Sequence
  Description of Artificial Sequence  note = synthetic construct
gaggggtcggaggtcttacccaacagattgacgcggcgttagtattggccgtgtacccgaaaaactgattgactgggctggcgttaactgtgcggaggcaggatttccctggggaaagtcttcactcagagcttcgctgacagccaaggatggcaagcaaggggccctcggcctctgcatctcctgagaactccagtgcaggggggcccagtgggagcagcaatggcgctggcgagagcggagggcaggacagcactttcgagtgcaacatctgcttggacacagccaaggatgccgtcatcagcctgtgtggccacctcttctgttggccgtgtttacatcagtggttggagaccagacctaacagacaggtgtgtcctgtttgcaaagctggcatcagccgagacaaggtcatccccctctatggaaggggcagcactgggcaacaggaccccagagagaagacccctcctcgtcctcaaggacagaggccagagccggagaatagagggggatttcaaggatttggatttggagatggtggcttccagatgtcttttggaattggggcatttccctttgggatatttgccacagcatttaatataaatgatgggcggcctcctccagctgtccctgggacaccccagtatgtggacgagcagttcctgtcacgcctcttcctatttgtggccctggtgatcatgttctggctcctgattgcctaaagctggggtcacttcgtgaattcaccagagactcaaagatcttttattggctctgggctgtgctcagtgtctttggcctcagagaacaacttgaatgacttcctggtttcctggcataaattattcctggtgagacatgtggcttaactcacaggtttcccatcagctttctccctaaaactatgttcatctgcctctctctgccagagaacatacagccgagaatactgccgaagctgagactgactactgtgcattaggaaagacctggagtcaggactttggtgggatttggagctccgaggcagtaataactgaacaagcagccctgtcccctaggctgcagaagcttgaatgcatcctctcccagaacctgccacaggaaactgggggctttgtcaggtcagcccaactgcatgcaaaagaccaccatcctcagaagccaagttgtcttttatgaagaggcaaggaaaggggaaacccacatgtgaccctgattttggtatggcttgatagagttccctgaaaactccttgtatgtgtgctaaaaccagggaagcatgtgactgccaagcaggcaacccctgatgatttgtaaagccaggtggcagggccttggggagccccagcacaatgatattgtgtggtcttccctcctgtggaatcgaggggaaattattcttcccaataccttgatttgattttcagtttcataagcttcttcctctgaatcttattgagggactatggtaccaagcaggtaggactgttcacctggtggaacagttcttgctctgccttctaggcttcatcccagaaatccagcctctttctggagaccccaaagctggagggagatgggctttcctctgggcctctctcctactttgccatccacactgctcctggctaaccccagcaataaccaacaaatggtaggaagccccatctattgctttttctcaattatgactgcatagtttatggaaacaaagatcttgaggaagatgagggaagccctcccctcttcacagtccccatctcttctcctttgtacctgtcaacaccagagttcagtgtttaaacagacaaaatataaagtattgagtaggtggttcatatgccgaatccacttggtaggaagaaaccaccagctttatagtgtgcctgatagattttaaacattcctgggcacccactcaagagtgctctttttatcaccttctggaaatccgcaaagttgcaggggcctctggagtgtctcttctctagagagaattggtgggacccccctcagtgcagtggccccaactagtggagggagagaggacttaagtcagatggactcaacagaaatgggtttccagaagaataatgaaaagttgtgggtaggaaaatgaatcatttggactcttcaatgaaatggagtgagcccaggagagctcagccaacagaggcactctgggaacctgttagtaaagccaggctggccaaatgccatttgattttgaacctcgtaggtccccactcaccctctgccaggagctaagtaaggcaggagagctgacttgggactcctggctcggccccaacagggagcccccttcccaccatccctcggcaagctcaccacctcatccttctgccaaggcagctttcctttcttttgtgtgttttctgtgttcttagcctccaccctcctcctgccacccttgtggactaggaccaggtcctgaccccagtcagaaaatgatgatatgtacagtggcacaccttaaccagtcactaattttcactgttgtgaaagtgatttgatttagaattaaacaaatggttttacattaaaaaaaaaaaaaaaaaaaaaaa

SEQ ID NO: 5 - protein, 192 aa, Artificial Sequence
  Description of Artificial Sequence  note = synthetic construct
MASKGPSASASPENSSAGGPSGSSNGAGESGGQDSTFECNICLDTAKDAVISLCGHLFCWPCLHQWLETRPNRQVCPVCKAGISRDKVIPLYGRGSTGQQDPREKTPPRPQGQRPEPENRGGFQGFGFGDGGFQMSFGIGAFPFGIFATAFNINDGRPPPAVPGTPQYVDEQFLSRLFLFVALVIMFWLLIA

SEQ ID NO: 6 - DNA, 27 bp, Artificial Sequence
  Description of Artificial Sequence  note = synthetic construct
gtacccatacgatgttccagattacgc

SEQ ID NO: 7 - DNA, 27 bp, Artificial Sequence
  Description of Artificial Sequence  note = synthetic construct
ctgagcagccagaaaaagaaaaagatg

SEQ ID NO: 8 - DNA, 24 bp, Artificial Sequence
  Description of Artificial Sequence  note = synthetic construct
cgggtctaccatcgagggcctgct

SEQ ID NO: 9 - DNA, 24 bp, Artificial Sequence
  Description of Artificial Sequence  note = synthetic construct
cccggggaatccccgtcccccaac

SEQ ID NO: 10 - DNA, 64 bp, Artificial Sequence
  Description of Artificial Sequence  note = synthetic construct
gatccccagctgggatcagcagagagttcaagagactctctgctgatcccagcttttttggaaa

SEQ ID NO: 11 - DNA, 21 bp, Artificial Sequence
  Description of Artificial Sequence  note = synthetic construct
ugucuucaucaguggcuggtt

SEQ ID NO: 12 - DNA, 21 bp, Artificial Sequence
  Description of Artificial Sequence  note = synthetic construct
guauguaaagcugggaucatt

SEQ ID NO: 13 - DNA, 21 bp, Artificial Sequence
  Description of Artificial Sequence  note = synthetic construct
gcugggaucagcagagagatt